(19) United States
(12) Patent Application Publication
(10) Pub. No.: US 2026/0265646 A1
(43) Pub. Date: Sep. 10, 2026

(54) PROCESS FOR THE PREPARATION OF AMINO ACID ESTERS AS ORGANOETHER SULFATE SALTS FROM ALKOXYLATED ALCOHOLS

(71) Applicant: BASF SE, Ludwigshafen am Rhein (DE)

(72) Inventors: Sophia EBERT, Ludwigshafen am Rhein (DE); Annalaura DEL REGNO, Ludwigshafen am Rhein (DE); Sean NATOLI, Cincinnati, OH (US)

(21) Appl. No.: 19/167,894

(22) PCT Filed: Mar. 21, 2024
(86) PCT No.: PCT/EP2024/057523

§ 371 Date: Sep. 23, 2025

(30) Foreign Application Priority Data
Mar. 24, 2023 (EP) 23164156.4
Mar. 27, 2023 (EP) 2316367.7
Mar. 27, 2023 (EP) 23164357.8

(51) Int. Cl. C11D 1/831; C07C 303/32; C11D 1/18; C11D 1/72; C11D 1/722; C11D 1/75; C11D 3/20; C11D 3/24; C11D 3/48

(52) U.S. Cl. CPC C11D 1/831; C07C 303/32; C11D 3/2068; C11D 3/24; C11D 3/48; C11D 1/18; C11D 1/72; C11D 1/722; C11D 1/75

(57) ABSTRACT

The invention relates to a process for the preparation of organoethersulfate salts of amino acids esters from alkoxylated alcohols, the ester salts, the ester, their use and compositions comprising such ester and/or ester salt.

The invention relates to a process for the preparation of organoethersulfate salts of amino acids esters from alkoxylated alcohols Organosulfate salts such as alkyl sulfate salts and alkylether sulfate salts are known to be water-soluble salts which are used as detergents or wettings agents.

Also, amino acids esters from alkoxylated alcohols and their salts are known as well.

FR2977585B1 discloses a process for the synthesis of α-amino acid esters of C₇- to C₃₆-alcohols from the amino acid or its salt in presence of an acidic catalyst such as sulfuric acid in the presence of water, starting from amino acids or salts thereof.

WO2015172158 discloses salts of ethanesulfonic acid alpha and higher amino acids esters. WO2011002746 discloses the preparation of amino acids esters with sulfuric acid

TRIVEDI, T. J. et al. ChemSusChem 2011, number 4, pages 604-608 describe a synthesis route to salts from alpha-C₃-C₄-amino acid esters and laurylsulfate which includes the formation of amino acid ester as hydrochloride salt, followed by ion exchange with sodium laurylsulfate.

SU1276661 discloses salts from protonated amino acid esters and anionic alkylsulfates. They are obtained from amino acids with 2 mol hexadecanol and excess sulfuric acid in dioxane.

JP49076822 and JP51036735 disclose a process for the preparation of amino acid ester salts with alkylsulfates by heating 1 mole of amino acid with at least 3 mole lauryl alcohol in presence of sulfuric acid. The synthesis is carried out in toluene as solvent.

Currently available methods for the synthesis of organosulfate salts of amino acids esters applying amino acids as starting materials are carried out in organic solvents and often involve an anion-exchange after the synthesis of the amino acid ester to replace the anion present in the esterification reaction with an organosulfate.

Similarly, WO 2019/007750 discloses esteramines from alkoxylated monoalcohols with amino acids which lists-among others-sulfuric acid as acid for the synthesis. However, no alkylether sulfates are obtained as counterions. The molar ratio of amino acid to hydroxyl groups of the alkoxylated alcohol of disclosed process step a) is 0.8:1 to 1:1.5. In one embodiment, the process is carried out with the molar ratio of the acid to the hydroxyl groups of the alkoxylated alcohol of step a) is in the range of from 0.1:1 to 1:1.

EP0847987 discloses a process, where esters from amino acids (or lactams) with C2-C40 linear, branched, or cyclic alcohols are obtained with sulfuric acid or phosphoric acid. Counterions possible are hydrogen sulfate or alkylsulfate. No information is given regarding the ratio of C2-C40 to sulfuric acid and amino acid, besides the minimum of 1:1:1. The process was carried out in presence of solvents or without solvent. No alkoxylated alcohols are described as reagents.

There is a continuous need for an improved process for the preparation of organoethersulfate salts of alkoxylated amino acids esters with high yield at fast reaction times, without handling of organic solvents and without handling of gaseous corrosive acids. There is also a need for a process for the preparation of organoethersulfates and alkoxylated amino acid esters in one reaction with high yield, reduced reaction time and complexity of the synthesis. There is also a need for a process which allows to react quantitatively amino acids and amino acid precursors such as lactams with alcohols which have a low water solubility and can therefore only be dispersed in water to obtain organoethersulfate salts of amino acids esters. All such processes should preferably employ a reduced amount of organic solvents, as those need to be removed prior to being able to employ them in cleaning compositions, and especially in such compositions for household uses.

Hence, there is a continuous need for providing alkoxylated amino acid ester salts and thus for their use combining an alkoxylated amino acid ester active with an organoethersulfate detergent for incorporation in improved detergent formulations for especially fabric and home care applications such as hard surface cleaning.

It was an object of the present invention to provide such a process which complies with the above identified objectives and needs.

This goal was achieved by the present invention as described herein below and as reflected in the claims and the examples.

### Definitions

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms. “Comprising” may be replaced in a preferred embodiment with “consisting essentially of” and both may be replaced by “consisting of” in an even more preferred embodiment.

The compositions of the present disclosure can “comprise” (i.e. contain other ingredients), “consist essentially of” (comprise mainly or almost only the mentioned ingredients and other ingredients in only very minor amounts, mainly only as impurities), or “consist of” (i.e. contain only the mentioned ingredients and in addition may contain only impurities not avoidable in an technical environment, preferably only the ingredients) the components of the present disclosure.

Similarly, the terms “substantially free of . . . ” or “substantially free from . . . ” or “(containing/comprising) essentially no . . . ” may be used herein; this means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 1%, or even less than 0.1%, or even more less than 0.01%, or even 0%, by weight of the composition.

Generally, as used herein, the term “obtainable by” means that corresponding products do not necessarily have to be produced (i.e. obtained) by the corresponding method or process de-scribed in the respective specific context, but also products are comprised which exhibit all features of a product produced (obtained) by said corresponding method or process, wherein said products were actually not produced (obtained) by such method or process. However, the term “obtainable by” also comprises the more limiting term “obtained by”, i.e. products which were actually produced (obtained) by a method or process described in the respective specific context.

When used herein any definition requiring a compound or a substituent of a compound to consist of “at least a number of carbon atoms”, number of carbon atoms refers to the total number of carbon atoms in said compound or substituent of a compound. For example for a substituent disclosed as “alkyl ether with at least 8 carbon atoms comprising alkylene oxide groups”, the total number of at least 8 carbon atoms needs to be the sum of the number of carbon atoms of the alkyl moiety and the number of carbon atoms of the alkylene oxide moieties.

All such terms not specifically defined have their ordinary meaning as known in the field of organic chemistry.

The term “containing one hydroxy group” means that only one group —OH is present. Any functionalized group derived from a hydroxy group such as an ether group is not considered to be an —OH group.

As used herein, the articles “a” and “an” when used in a claim or an embodiment, are understood to mean one or more of what is claimed or described. As used herein, the terms “include(s)” and “including” are meant to be non-limiting, and thus encompass more than the specific item mentioned after those words.

The term “about” as used herein encompasses the exact number “X” mentioned as e.g. “about X %” etc., and small variations of X, including from minus 5 to plus 5% deviation from X (with X for this calculation set to 100%), preferably from minus 2 to plus 2%, more preferably from minus 1 to plus 1%, even more preferably from minus 0.5 to plus 0.5% and smaller variations. Of course if the value X given itself is already “100%” (such as for purity etc.) then the term “about” clearly can and thus does only mean deviations thereof which are smaller than “100”.

The term “free of water” means that the composition contains no more than 5 wt.-% of water based on the total amount of solvent, in another embodiment no more than 1 wt.-% of water based on the total amount of solvent, in a further embodiment the solvent contains no water at all.

All temperatures herein are in degrees Celsius (C) unless otherwise indicated. Unless otherwise specified, all measurements herein are conducted at 20° C. and under the atmospheric pressure. In all embodiments of the present disclosure, all percentages are by weight of the total composition, unless specifically stated otherwise. All ratios are weight ratios, unless specifically stated otherwise.

The phrase “fabric care composition” is meant to include compositions and formulations designed for treating fabric. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, unit dose formulation, delayed delivery formulation, detergent contained on or in a porous substrate or nonwoven sheet, and other suitable forms that may be apparent to one skilled in the art in view of the teachings herein and detailed herein below when describing the compositions. Such compositions may be used as a pre-laundering treatment, a post-laundering treatment, or may be added during the rinse or wash cycle of the laundering operation, and as further detailed herein below when describing the use and application of the inventive and compositions comprising such esteramine and its salts.

The term “containing one hydroxy group” means that only one group-OH is present. Any functionalized group derived from a hydroxy group such as an ether group is not considered to be an —OH group.

Throughout this description, the term “inventive compound” may be used instead of the “inventive esteramine(s) and/or their salt(s)” and “esteramine(s) and/or their salt(s) of this (present) invention”, meaning those compounds being disclosed herein as invention, defined by their structure and/or their process to produce or obtainable by the process defined herein. Similarly, “inventive process” means the process disclosed and defined herein.

Preferably, “inventive compound” means the organoethersulfate salts of the aminoacid esters as defined herein by their process to produce and their structures resulting from such process.

### DETAILED DESCRIPTION

The definitions and their preferences given within the “Definition”-section before are included as part of this invention as described herein below.

The specific embodiments as described throughout this disclosure are encompassed by the present invention as part of this invention; the various further options being disclosed in this present specification as “optional”, “preferred”, “more preferred”, “even more preferred” or “most preferred” (or “preferably” etc.) options of a specific embodiment may be individually and independently (unless such independent selection is not possible by virtue of the nature of that feature or if such independent selection is explicitly excluded) selected and then combined within any of the other embodiments (where other such options and preferences can be also selected individually and independently unless such independent selection is not possible by virtue of the nature of that feature or if such independent selection is explicitly excluded), with each and any and all such possible combinations being included as part of this invention as individual embodiments.

### Process

The present invention relates to a process for the synthesis of organoethersulfate salts of alkoxylated amino acid esters from alkoxylated mono-alcohols and alpha-amino acids with alkylether sulfate counterion.

The inventive compounds can be obtained from reaction of at least 2 moles of alkoxylated mono-alcohols with 1 mole of alpha-amino acid and 1 mole of sulfuric acid in the presence of water.

The general structure of the inventive compounds are shown in formula (I) below.

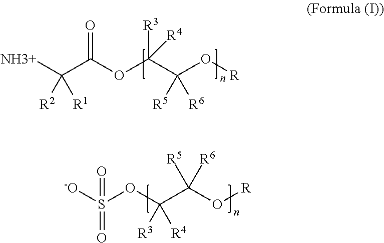

with
n=1-200
R=C1-C36-alkyl or -alkenyl or -phenyl or -alkylphenyl
R1, R2=hydrogen or C1-C12-alkyl or -phenyl
R3, R4, R5, R6=hydrogen or C1-C 20-alkyl

Hence, the present invention relates to a process for the synthesis of organoethersulfate salts of alkoxylated amino acid esters comprising the steps of
(i) reacting at least one alpha-amino acid, and at least one alkoxylated alcohol with sulfuric acid in an aqueous solution; and
(ii) optional removal of water and/or removal of excess alcohol.

More specifically, the present invention relates to a process to produce organoethersulfate salts of alkoxylated amino acid esters comprising the steps of
(A) providing at least one alcohol bearing one hydroxy group, wherein such hydroxy group is alkoxylated with at least one alkylene oxide moiety, preferably at least 1 and up to 200, more preferably 1 to 100, more preferably up to 50, even more preferably up to 20, even more preferably up to 20, even more preferably up to 15, and most preferably up to 10 moles alkylene oxide per hydroxy group,
(B) providing at least one alpha-amino acid, and
(C) sulfuric acid,
wherein the molar ratios are at least as follows:
   the ratio between amino acid to hydroxyl groups of the alkoxylated alcohol is minimum 1:2 or 1:(greater than 2); and
   the ratio of sulfuric acid to amino acid is 0.9:1 to 1.1:1 and
   the ratio between hydroxyl groups of alkoxylated alcohol and sulfuric acid is minimum 2:1 and preferably (>2): 1;
   the molar ratio of water to amino acid is from 0.5:1 to 10:1, preferably up to 5:1, more preferably up to 2:1, and most preferably up to 1:1, such as about 1:1 and exactly 1:1;
(D) Reacting the at least one alkoxylated alcohol with the at least one alpha-aminoacid in the presence of sulfuric acid to obtain the organoethersulfate salts of alkoxylated amino acid esters,
(E) Optionally remove water and/or excess alcohol during and optionally also after the reaction to obtain the purified organoethersulfate salts of alkoxylated amino acid esters.

Further process steps can be included in the inventive process:

The reaction mixture may be inertized before and/or during the reaction, preferably prior to the addition of the sulfuric acid.

Inertization can be achieved by a steam of gas such as nitrogen or argon, and/or by adding an oxygen-scavenging compound.

Such oxygen-scavengers are known. Advantageous is the use of small amounts of hypophosporous compound, such as hypophosphorous acid or any of their salts, especially any alkali or ammonium or earth alkali salts, more preferably the acid, which can be added to the reaction mixture prior to adding the sulfuric acid.

As one of the effects, this addition of the oxygen-scavenging compound leads to an improved colour of the resulting product, i.e. meaning a reduced coloring, when compared to omitting such addition.

The reaction is performed the reaction is performed
at a temperature of from 50 to 200° C., preferably 100-200° C., more preferably 120° C.-180° C., most preferably 120° C.-150° C., such as 60, 70, 80, 90, 110, 115, 120, 125, 130, 135, 140, 145, 160, 170, 190° C.;
for a period of from 1 to 30, preferably from 2, more preferably from 3 hours, and preferably up to 20, more preferably up to 165, even more preferably up to 10 hours, such as preferably 3 to 24, more preferably 5 to 24, most preferably 10-24 hour(s); and
at from 0.001 to 10 bar pressure, such as from 0.1, more preferably from 1, and preferably up to 8, more preferably up to 5, even more preferably up to 4 bar, such as 1 to 10, more preferably 1 to 5, even more preferably 1 to 4 bar.

Preferably, the temperature, the duration and the pressure are all as defined individually before, preferably the preferred ranges of all three parameters, more preferably the more preferred ranges of all three parameters and so on are selected.

In one embodiment of the present invention, the temperature is kept constant for the duration of the reaction. In another embodiment, the temperature is varied within the temperature range during the duration of the reaction.

In one embodiment of the present invention the reaction with sulfuric acid is carried out under atmospheric pressure. In another embodiment the reaction with sulfuric acid is carried out in a closed vessel under a pressure of from 0.001 to 1 bar. In another embodiment the reaction with sulfuric acid is carried out in a closed vessel under pressure of more than 1 to 10 bar.

In one embodiment a protective atmosphere of an inert gas such as nitrogen gas or argon gas is used to carry out the reaction.

In another embodiment inertization is carried out by adding an oxygen-scavenging agent, preferably a hypophosphorous compound, more preferably hypophosphorous acid.

In another embodiment inertization is carried out by adding an oxygen-scavenging agent as defined before and with the use of a protective atmosphere of an inert gas as defined before.

It is clear that the temperature, pressure and duration can be chosen independently from the disclosed values and ranges and combined with each other. Similarly, to this also the way inertization is achieved can be chosen and combined with the temperatures, durations and pressures chosen, all as defined before.

During or following the reaction, preferably at least during the reaction, water and/or excess alcohol can be removed. Removal of water and alcohol can be carried out by all techniques known in the art, for example by application of a stream of gas and/or applying a distillation method, preferably a distillation, more preferably a distillation method under reduced pressure and/or at elevated temperature, preferably both, a preferred method being the use of an apparatus such as a Dean-Stark-trap. In another embodiment, water and/or alcohol is removed using a stream of gas, such as using gas such as inert gas as nitrogen or argon, preferably nitrogen, or steam made from water, preferably using inert gas, more preferably nitrogen.

In a more preferred embodiment, water and/or alcohol is removed by application of a vacuum and/or increasing the temperature, most preferably using an apparatus such as a Dean-Stark-trap. The optional removal of water and/or excess of alcohol is more preferably carried out applying a vacuum in the range of from 0.1 mbar to 800 mbar, preferably of from 1 mbar to 500 mbar and more preferably of from 10 mbar to 100 mbar, and using elevated temperatures.

In a preferred embodiment, during the reaction a steam of inert gas, such as nitrogen or argon, preferably nitrogen, is passed through the reaction mixture. This helps to keep the reaction mixture inert, but also supports the removal of forming water from the chemical reaction, and thus to control the amount of water present.

The reaction is performed starting from a suspension of the typically solid aminoacid(s) in the alkoxylated alcohol(s). Typically, no organic solvent is added nor present at the beginning besides the alkoxylated alcohol(s) and water. During the course of the reaction the product formed is soluble and thus the reaction mixture turns towards becoming a solution, with the reaction mixture typically ending up as a homogeneous solution of the inventive compound(s) in alkoxylated alcohol containing residual amounts of water—with the content of alkoxylated alcohol and water depending on the if and the how the process measure of removing such alcohols and/or water as defined herein elsewhere is performed.

Such “residual amounts of water” may be below 5 percent, preferably below 2, and—typically and thus even more preferably-below 1 percent based on the total weight of the reaction mixture.

The sulfuric acid employed typically contains some water. In one embodiment, sulfuric acid is used as concentrated sulfuric acid. In another embodiment, sulfuric acid is used as 96 to 98 wt.-% sulfuric acid solution in water. In a further embodiment sulfuric acid is used as 80 wt.-% sulfuric acid solution in water.

In one embodiment, no further water is introduced into the reaction as such besides the water being introduced within the sulfuric acid, the aminoacid(s) and the alkoxylated alcohol(s).

In a preferred embodiment, the amount of water (which is present at the start of the reaction) per one mole of aminoacid is from 0.1:1 to 10:1, preferably up to 5:1, more preferably up to 2:1, and most preferably up to 1:1, and preferably from 0.5:1; more preferably from 0.7:1, such as about 1:1 and exactly 1:1.

Preferably, no further organic solvent is introduced into the reaction as such besides the organic solvents possibly being present within any of the starting materials employed, i.e. within the sulfuric acid, the aminoacid(s) and the alkoxylated alcohol(s).

In one embodiment, the amino acid, the alkoxylated alcohol, water and the optional oxygen-scavenging agent is mixed into a solution or emulsion, preferably solution. Such solution is obtained when the ingredients all are soluble. The amount of water may be adjusted to improve dissolution, the amount of water being within the ranges as defined herein elsewhere.

Typical concentration of amino acid in the reaction mixture is in the range of from 1% by weight to 99%, preferably 5-50%, more preferably 5-30%, most preferably 10-25%. by weight based on the total weight of aminoacid, alkoxylated alcohol and water. The amount also depends of course on the relative molecular weights of the reactants; the higher the molecular weight of e.g. the alkoxylated alcohol, the lower the relative amount of the amino acid is typically chosen, to compensate e.g. for viscosity effects.

Preferably, the alkoxylated alcohol(s) serve as solvent for the reaction, with the amounts of water being introduced or present in the reaction mixture at the start of the reaction is as defined herein elsewhere.

In one embodiment of the present invention the total amount of sulfuric acid is added at the beginning of the reaction to the at least one amino acid and alkoxylated alcohol. In another embodiment the sulfuric acid is added dropwise for a duration of from 0.1 and up to 15, preferably up to 10, more preferably up to 5 hours, to the reaction mixture comprising the at least one alkoxylated alcohol and the at least one amino acid. Typically, the duration of the addition of the sulfuric acid is not more than 5, preferably not more than 3, even more preferably not more than 2 hours, and may be as shott as up to 60, 45, 30 or even 15 minutes only.

In another, preferred embodiment the duration of the sulfuric acid addition is about at most 10% of the total reaction time, more preferably at most 5%.

Of course, it is clear that the time of addition of the sulfuric acid also depends in typical known ways on the scale of the reaction and the overall duration of the reaction.

In another embodiment, the sulfuric acid is added to the mixture of alkoxylated alcohol(s), aminoacid(s) and optionally water. The reaction mixture can be at an elevated temperature or at ambient temperature, such as room temperature, when the sulfuric acid addition is started. During the course of this addition, the temperature of the reaction mixture can rise due to release of energy from the chemical reaction. During or after addition of the sulfuric acid, the temperature of the reaction mixture can be raised to the reaction temperatures as defined herein elsewhere, preferably after.

The reaction compartment typically- and preferably—is equipped with a condenser to control reflux. Reflux can be also controlled by increasing the pressure within the reaction compartment when choosing appropriate temperatures and corresponding pressures from the ranges as defined herein elsewhere.

In one embodiment of the present invention the reaction is carried out by mixing at least one alkoxylated alcohol and at least one amino acid and water to obtain an aqueous solution or aqueous suspension, optionally adding at least one oxygen-scavenging agent as herein defined before and/or inertization by preferably applying an inert gas such as providing such gas by preferably bubbling through the reaction mixture, then adding the sulfuric acid, optionally followed by sealing of the reaction vessel and optionally applying pressure as defined herein elsewhere, heating the mixture at a temperature as detailed herein elsewhere, reacting for a duration as detailed herein elsewhere, and at a pressure as detailed herein elsewhere, thereby reacting the mixture to obtain the alkoxylated esteramine sulfate salts as defined in this invention.

organoethersulfates are esters derived from alcohols and sulfuric acid. Organoethersulfates comprise the group “R—O—SO₃—”, comprising a “—O—SO₃”-core and a R-group which may be selected from the group consisting of linear alkyl, branched alkyl, linear alkylether, branched alkylether, and phenoxyalkyl.

Alcohols containing one hydroxy-group are well-known in the art. The respective alcohol may contain linear, branched and/or cyclic alkyl fragments. Beyond that, the respective alcohol may also contain aromatic fragments as well as combinations of alkyl and aromatic fragments (“aralkyl” fragments). Furthermore, the respective alcohol may also contain alkyl ether fragments.

The alkoxylated alcohol (A) for the alkoxylated amino acid esters and salt thereof is an alcohol bearing one hydroxy-group which can react during the inventive process. The alkoxylated alcohol however may be derived from alcohols bearing more than one hydroxy-group, provided however that such higher alcohols are alkoxylated and modified in such way that only one hydroxy-group per alkoxylated alcohol compound remains free for reaction in the inventive process. This can be done for example by modifying (often termed also “end-capping”) any further hydroxy-group being present to yield an alkoxylated monoalcohol having only one hydroxy-group present before the present reaction is performed.

The alkoxylated monoalkohol is preferably selected from
alkoxylated mono-alcohols such as C1- to C36-alkanols, selected from the group comprising linear C2- to C36-alcohols, such as mixture of such alcohols selected from C6- to C22-fatty alcohols, preferably C8- to C22-fatty alcohols, more preferably C12- and C14-fatty alcohols, most preferably C16- and C18-fatty alcohols; branched C3- to C36-alcohols such as 2-ethylhexanol, 2-propylheptanol, isotridecanol, isononanol, C9-C17 oxoalcohols;
alkoxylated branched C3- to C36-alcohols such as alkoxylated 2-ethylhexanol, alkoxylated 2-propylheptanol, alkoxylated isotridecanol, alkoxylated isononanol, alkoxylated C9-C17 oxoalcohols;
and alkoxylated phenoxyalkanols such as alkoxylated phenoxyethanol;
with the alcohols preferably selected from the group alkoxylated linear mono-alcohols C2- to C22-alcohols, most preferably an alkoxylated linear C12-C14-mono alcohol and/or alkoxylated 2-propylheptanol;
with the alkoxylation and any end-capping of further hydroxy-groups optionally being present before-such that in effect the alkoxylated alcohol contains only one hydroxy-group although the alcohol or the alkoxylated alcohol might have contained two or more hydroxy-groups before end-capping of all hydroxy-groups except one—to be performed using standard means and prior to the inventive process.

Obviously, such alcohols are known, many of them from natural sources. Others can be produced starting from natural sources by modifying them. If such modification does result in the addition of carbon-atoms, e.g. by chemical addition reactions, and if such addition reactions use non-fossil carbon atoms, then alcohols contain only non-fossil based carbon. Hence, it is preferred, that only naturally occurring alcohol and/or nature-derived alcohols (not being made with the addition of non-fossil carbon in building blocks to obtain such nature-derive alcohols) are employed in this invention. Even more preferably, only naturally occurring alcohols are employed.

The alcohol (A) employed for the alkoxylated amino acid esters and salt thereof produced according to the inventive process is an alkoxylated alcohol which is obtained by alkoxylating the hydroxy group of the alcohol with one or more alkylene oxides to produce alkylene oxy-chains having an free hydroxy-group at the end, such chains comprising one or more moieties stemming from alkylene oxides selected from C2 to C22-alkylene oxides, preferably C2-C4-alkylene oxides, whereas the moieties stemming from the alkylene oxide(s) may be arranged in random, block or multiblock-order or combinations thereof, preferably as block.

The alkoxylation of the alcohol can be achieved by either carrying out the alkoxylation reaction with only one alkylene oxide or with more than one alkylene oxide. If more than one alkylene oxide is used, the resulting alkylether alcohols comprises either randomly distributed alkylene oxide units or a block of one alkylene oxide followed by a block of another alkylene oxide or a block of one alkylene oxide followed by another block which comprises two or more alkylene oxides arranged in random order or a block comprising two or more alkylene oxides is followed by another block which comprises two or more alkylene oxides with each such block being different in their relative amount of alkylene oxides, their arrangement of alkylene oxides and/or the identity of the alkylene oxides such that the two blocks linked to each other differ in their chemical composition and/or arrangement; any such combination of arrangements is in principle possible, and as such is encompassed by this present invention.

In one embodiment of the present invention, alkyl alcohols alkoxylated with only a single alkylene oxide are used. In a further embodiment, alkyl alcohols alkoxylated with a first alkylene oxide followed by alkoxylation with a second alkylene oxide, thereby forming a block structure of different alkylene oxide blocks, are used.

It is clear that when switching from one block to the next there might be—depending on how the alkoxylation reaction is performed—there might be a relatively small “region” in between the blocks which do not have a sharp “border” between the block which is identical on every molecule of the alkoxylated monoalkohol, such that there might be some “dirty” structures which means that some very small amounts (such as a single or perhaps two or three) alkylene oxides of the first type employed might be inserted only after one or more of the second alkylene oxide have reacted; this depends mainly on the conversion rate achieved for the first alkylation reaction at the time point when the second alkylene oxide is added, i.e. if at that time of adding the second alkylene oxide still some unreacted amounts of the first alkylene oxide are present.

Similarly, it is clear that due to the statistical reaction of the alkoxylation polymerization the block lengths might differ slightly from individual compound to the next, and thus the “the alkoxylated mono-alcohol” in fact is a mixture of compounds having slightly differing chain lengths and block lengths.

Moreover, it is clear to a person of skill in the art, that the definition of the inventive compounds according to the formula(s) provided herein is a theoretical result of an optimized way for carrying out the respective reaction and process, wherein all functional groups (of the respective starting material or any intermediate) have undergone a complete reaction. It is also clear, however, that a complete reaction (the conversion degree of 100%) is an idealized assumption. In reality, the degree of conversion is usually below 100%. Unreacted hydroxy groups etc. may be present. This fact is known to a person skilled in the art for such reactions as well as the structure according to the formulas. Irrespective of that, the reaction for obtaining said structure is disclosed in the description above. By following the general reaction conditions as well as the specific reaction conditions detailed herein including the example section, the real structure for each individual case/reaction condition is obvious for a person skilled in the art or can be determined using standard methods.

For the sake of completeness, it is mentioned in connection with the present invention, especially in connection with the esteramines according to formula disclosed herein and even more so the alkoxylation reactions leading to the alkoxylated alcohols employed for the present reaction, that the reactions employed and especially the alkoxylation reaction towards the alkoxylated alcohols are known by a person of skill to be leading to statistical distributions. This means that when for example “20 ethylene oxide (“20 EO”) units per functional group of the molecule (i.e. in the following for simplicity termed the “core”) to be reacted” are employed, that does not necessarily mean that each such functional group of the core actually will bear exactly 20 EO-units; to the contrary, the resulting product obtained from such reaction is a mixture of various slightly differing products, with the main product contained in the product of the process being the product having a core being modified on each functional group with exactly 20 EO-units per functional group; however, due to the statistical reasons, this “main product” (in the present case the “alkoxylated monoalkohol”) is accompanied by many products having slight variations to this main product, where e.g. the same core-molecule is modified with EO but the lengths of the EO-chains per functional group slightly differing from 20: some chains are slightly longer and some slightly shorter, a typically even smaller amount bearing even more EO and some even less EO, an even smaller amount differs to a larger extent etc. The more functional groups the core might bear (e.g. the more hydroxy-groups an alcohol bears), and the more e.g. alkylene oxide-units per functional group are employed, the larger is the overall deviation from the “targeted” molecule depicted via such formulas: this means, that the content of the targeted molecule (which is depicted in the formula(s) herein) decreases within the product mixture obtained (i.e. such content is then below 100% of the total amount of compound obtained), and more different, slight variations of the targeted molecule are present in addition to the targeted molecule.

This however does not cause any problem, as the explanation herein for such reactions is the identical “problem” observed for each and every polymerization reaction: Every polymer being prepared is defined by a chemical structure. Depicting such polymer structure, however, is as difficult as for the structures contemplated in this present invention: the more precise the structure of a polymer is defined, the more this depicted structure is wrong. Hence, polymers are being described by the monomers they are created from, the reaction employed (e.g. “by radical polymerization” which then implies how the monomeric units are linked), and certain other typical values such as the molecular weight Mw, Mn, the polydispersity index (i.e. the broadness of the molecular weight distribution) etc.—which in fact is nothing else than saying that the polymer product obtained from the polymer reaction is a mixture of various polymer molecules which have different chain lengths, slightly differing orders of monomeric units within a chain (if more than one monomer is employed), slightly differing amounts of each monomeric unit within a chain (if more than one monomeric unit is employed) and so on.

For the present structure contemplated in this present invention, the same difficulty can arise as explained for a polymer molecule description but complicated with the fact, that a defined organic molecule/molecules is/are also employed: the molecule(s) is/are a clearly chemically defined “organic structure”, which can be pinned down exactly. However, the modifications through the inventive reaction herein are introduced by addition reactions with usually incomplete conversion, thus introducing the concept which is the same as for description of polymers (and their “relative description” via starting materials, i.e. monomers) into a—by formula—seemingly clearly defined organic molecule. This means for the presently contemplated structure that although it seems like a clearly defined structure of a “typical organic molecule”, this is in fact not necessarily the case: the present structure is a combination of clearly defined organic chemical structure fused together with “polymer descriptions” of the alkoxylated parts (when an alkoxylated alcohol is employed; such parts which are oligomers or polymers-depending on the amount of monomeric units employed for the alkoxylation) and also depending on how complete the various esterifications proceed.

This has to be taken in mind when the present structure is defined: the “organic structural parts” (the amino acid-structural part) can be easily and clearly defined in terms of organic chemistry, whereas the “polymer structural parts” (i.e the alkoxylated parts and the structures formed via esterification) seem-on paper—to be also following the “organic chemicals structure description”, but in fact should be viewed also with the eye of a polymer chemist.

With this in mind, it is clear that the “structure of a formula (X)” is a combination of organic chemistry description for the starting materials employed and a polymer chemistry-description for the alkoxylated parts and the chains resulting from esterification reactions and thus the “target molecule” of the reaction but not a “100%-structure” as in other organic molecules: The structure shown is the “main component” of the process described, the product containing smaller amounts of many slight variations of this main component side-by-side as explained herein before.

Within the context of the present invention, it is also preferred that in case compound (A) comprises an alkoxylated alcohol comprises the alkoxylated fragment being based on at least one C2-C22 alkylene oxide, preferably C2-C4-alkylene oxides, more preferably on ethylene oxide and/or propylene oxide, most preferably the respective alcohol comprises at least one block based on ethylene oxide and/or propylene oxide, and even more preferably contains only one block consisting of ethylene oxide or consisting of two blocks with the first block—preferably the “inner block” directly linked to the hydroxy-group of the alcohol—consisting of ethylene oxide and a second block—preferably being the “outer block linked to the ethylene oxide-block—consisting of propylene oxide. More preferably, the EO-block comprises at least 2 EO-derived moieties and the PO-block comprises at least 2 PO-derived moieties; the overall amounts, preferred amounts etc for AO apply as defined before.

It is noted that the alkylene oxide used to prepare the alkoxylated alcohol(s) may be derived from a fossil or non-fossil carbon source or even a mixture thereof. Preferably, the amount of non-fossil carbon atoms in the alkylene oxide employed is at least 10%, at least 20%, at least 40%, at least 70%, at least 95% and most preferably up top 100% based on non-fossil derived carbon atoms; the same applies to the total inventive compound as such. The skilled person is well-aware of commercial alkylene oxide products made of non-fossil carbon sources (these products are often sold as being “sustainable”, “renewable” or “bio-based”). For example, Croda International, Snaith, UK, sells ethylene oxide and related products based on bio-ethanol as “ECO”-Range. Additionally, methods to prepare bio-based propylene oxide are also known (see Abraham, D. S., “Production of propylene oxide from propylene glycol” Master's Thesis University of Missouri-Columbia (2007) (75 pages)).

The amino acid can be in principle any known amino acid. Preferably, the at least one amino acid is selected from any alpha-amino acid such as alanine, glycine, leucine, isoleucine, valine, proline, phenylalanine, arginine, asparagine, aspartic acid, aspartate, glutamine, glutamate, histidine, lysine, threonine, tryptophan, tyrosine, cysteine, methionine, serine; alpha-amino acids with secondary or tertiary amino groups such as sarcosine, N,N-dimethylglycine;
preferably alanine, valine, glycine, leucine, isoleucine, phenylalanine, more preferably alanine, valine, leucine, most preferably alanine and valine.

Obviously, such aminoacids are known, usually from natural sources. Others can be produced starting from natural sources by modifying them to obtain aminoacids as starting material in the present invention. If such modification does result in the addition of carbon-atoms, e.g. by chemical addition reactions, and if such addition reactions use non-fossil carbon atoms, then aminoacids contain only non-fossil based carbon. Hence, it is preferred, that only naturally occurring aminoacids and/or nature-derived aminoacids (not being made with the addition of non-fossil carbon in building blocks to obtain such nature-derived aminoacids) are employed in this invention. Even more preferably, only naturally occurring aminoacids are employed.

When the salts of the inventive aminoesters are neutralized using known means, then of course also the neutral aminoacid esters can be obtained from the present inventive process.

The present invention can also provide inventive amino acid esters and their salts starting from natural amino acids and/or natural alcohols, and thus enables the provision of products with high content of bio-derived material. As the alkylene oxide(s) can be also derived from non-fossil sources, the inventive compound can be obtained containing only carbon-atoms from non-fossil sources. Such products are being highly sought after by consumers and industries.

### Uses

The inventive alkoxylated amino acid esters and especially the organoethersulfate salts as directly obtained from the inventive process (without later desalination to obtain the neutralized aminoacid esters) can be used advantageously in cleaning compositions.

They may be used as at least one aminoacid ester, at least one organoethersulfate salt of at least one aminoacid ester, or any combination thereof.

Hence, another subject matter of the present invention is the use of the above-mentioned alkoxylated amino acid esters and/or their salts in cleaning compositions, specifically as prepared by the process defined herein.

The alkoxylated amino acid esters and/or their salts can be added to cleaning compositions. The alkoxylated amino acid esters and/or their salts are present in general in said formulations at a concentration of from about 0.1% to about 50%, preferably from about 0.25% to 15%, more preferably from about 0.5% to about 10%, and even more preferably from about 0.5% to about 5%, and most preferably in amounts of up to 3%, each in weight % in relation to the total weight of such composition/product, optionally further comprising from about 1% to about 70% by weight of a surfactant system, wherein-specifically—for a liquid hand dishwashing or spray detergent cleaning composition such composition comprising from 0.1% to 50%, preferably from 1% to 35%, more preferably from 3% to 30%, by weight of the total composition, of a surfactant system, and such surfactant system preferably comprising from 60% to 90%, more preferably from 70% to 80% by weight of the surfactant system of an anionic surfactant.

Hence, another subject matter of the present invention is the use of the alkoxylated amino acid esters and/or their salts obtained by a process of the invention as detailed before, in fabric and home care products, in particular cleaning compositions for improved oily and fatty stain removal, removal of solid dirt such as clay, prevention of greying of fabric surfaces, and/or anti-scale agents, wherein the cleaning composition is preferably a laundry detergent formulation and/or a dish wash detergent formulation, more preferably a liquid laundry detergent formulation and/or a liquid manual dish wash detergent formulation.

Another subject-matter of the present invention is, therefore, also a cleaning composition, fabric and home care product, industrial and institutional cleaning product, preferably in laundry detergents, in cleaning compositions and/or in fabric and home care products, each comprising at least one alkoxylated amino acid esters and/or their salts obtained by a process of the invention.

A further subject-matter of the present invention is a fabric and home care product, cleaning composition, industrial and institutional cleaning product, preferably a laundry detergent, a cleaning composition and/or a fabric and home care product, each containing at least one alkoxylated amino acid esters and/or their salts obtained by a process of the invention.

In a preferred embodiment, it is a cleaning composition and/or fabric and home care product and/or industrial and institutional cleaning product, comprising at least one alkoxylated amino acid esters and/or their salts obtained by a process of the invention. In particular, it is a cleaning composition for improved cleaning performance, especially improved primary washing, preferably a laundry detergent formulation and/or a manual dish wash detergent formulation, more preferably a liquid laundry detergent formulation and/or a liquid manual dish wash detergent formulation.

In a preferred embodiment, the cleaning composition of the present invention is a liquid or solid laundry detergent composition, preferably a liquid laundry detergent composition.

In another preferred embodiment, the cleaning composition of the present invention is a liquid or solid (e.g. powder or tab/unit dose) detergent composition for manual or automatic dish wash, preferably a liquid manual dish wash detergent composition. Such compositions are known to a person of skill in the art.

In another embodiment, the cleaning composition of the present invention is a hard surface cleaning composition that may be used for cleaning various surfaces such as hard wood, tile, ceramic, plastic, leather, metal, glass.

In one embodiment of the present invention, the inventive alkoxylated amino acid esters and/or their salts obtained by a process of the invention is a component of a cleaning compositions or fabric and home care product, preferably a laundry cleaning composition, a laundry care product or laundry treatment product or laundry washing product, preferably a liquid laundry detergent formulation or liquid laundry detergent product, that each additionally comprise at least one surfactant, preferably at least one anionic surfactant.

In one embodiment it is also preferred in the present invention that the cleaning composition comprises (besides at least one alkoxylated amino acid esters and/or their salts obtained by a process of the invention) additionally at least one enzyme, preferably selected from one or more optionally further comprising at least one enzyme, preferably selected from one or more lipases, hydrolases, amylases, proteases, cellulases, hemicellulases, phospholipases, esterases, pectinases, lactases, pectate lyases, cutinases, DNases, xylanases, oxicoreductases, dispersins, mannanases and peroxidases, and combinations of at least two of the foregoing types, preferably at least one enzyme being selected from lipases.

Even more preferably, the cleaning compositions of the present invention comprising at least one inventive alkoxylated amino acid esters and/or their salts obtained by a process of the invention and optionally further comprising at least one surfactant or a surfactant system—as detailed before—are those for improved cleaning performance within laundry and manual dish wash applications, even more specifically, for improved cleaning performance (such actions as detailed before) such as those on fabrics and dishware, and may additionally comprise at least one enzyme selected from the list consisting of optionally further comprising at least one enzyme, preferably selected from one or more optionally further comprising at least one enzyme, preferably selected from one or more lipases, hydrolases, amylases, proteases, cellulases, hemicellulases, phospholipases, esterases, pectinases, lactases, pectate lyases, cutinases, DNases, xylanases, oxidoreductases, dispersins, mannanases and peroxidases, and combinations of at least two of the foregoing types, preferably selected from one or more lipases, hydrolases, amylases, proteases, cellulases, and combinations of at least two of the foregoing types, more preferably at least one enzyme being selected from lipases.

In one embodiment, the inventive alkoxylated amino acid esters and/or their salts obtained by a process of the invention may be utilized in cleaning compositions comprising a surfactant system comprising C10-C15 alkyl benzene sulfonates (LAS) as the primary surfactant and one or more additional surfactants selected from non-ionic, cationic, amphoteric, zwitterionic or other anionic surfactants, or mixtures thereof.

In a further embodiment the inventive alkoxylated amino acid esters and/or their salts obtained by a process of the invention may be utilized in cleaning compositions or fabric and home care product, preferably a laundry cleaning composition, a laundry care product or laundry washing product, preferably a liquid laundry detergent formulation or liquid laundry detergent product, comprising C12-C18 alkyl ethoxylate surfactants with 5-10 ethoxy-units as the primary surfactant and one or more additional surfactants selected from anionic, cationic, amphoteric, zwitterionic or other non-ionic surfactants, or mixtures thereof.

In a further embodiment, the inventive alkoxylated amino acid esters and/or their salts obtained by a process of the invention may be utilized in the cleaning compositions or fabric and home care product, preferably a laundry cleaning composition, a laundry care product or laundry treatment product or laundry washing product, preferably a liquid laundry detergent formulation or liquid laundry detergent product, comprising C8-C18 linear or branched alkyl ethersulfates with 1-5 ethoxy-units as the primary surfactant and one or more additional surfactants selected from non-ionic, cationic, amphoteric, zwitterionic or other anionic surfactants, or mixtures thereof.

In one embodiment of the present invention, the alkoxylated amino acid esters and/or their salts obtained by a process of the invention is a component of a cleaning composition, such as preferably a laundry or a dish wash formulation, more preferably a liquid laundry or manual dish wash formulation, that each additionally comprise at least one surfactant, preferably at least one anionic surfactant.

In a further embodiment, this invention also encompasses a composition comprising at least one alkoxylated amino acid esters and/or their salts obtained by a process of the invention, further comprises an antimicrobial agent as disclosed hereinafter, preferably selected from the group consisting of 2-phenoxyethanol, more preferably comprising said antimicrobial agent in an amount ranging from 2 ppm to 5% by weight of the composition; even more preferably comprising 0.1 to 2% of phenoxyethanol.

In a further embodiment, this invention also encompasses a composition, preferably a cleaning composition, more preferably a liquid laundry detergent composition or a liquid hand dish composition, even more preferably a liquid laundry detergent composition, or a liquid softener composition for use in laundry, such composition comprising an alkoxylated amino acid esters and/or their salts obtained by a process of the invention in the amounts detailed before as described herein before, such composition further comprising 4,4′-dichloro 2-hydroxydiphenylether in a concentration from 0.001 to 3%, preferably 0.002 to 1%, more preferably 0.01 to 0.6%, each by weight of the composition.

In a further embodiment, this invention also encompasses a composition, specifically a cleaning composition, more preferably a cleaning composition in liquid, solid or semi-solid form, preferably being a concentrated liquid detergent formulation, single mono doses laundry detergent formulation, liquid hand dish washing detergent formulation or solid automatic dish washing formulation, more preferably a laundry detergent formulation, comprising an alkoxylated amino acid esters and/or their salts obtained by a process of the invention and in the amounts as detailed before, such composition being preferably a detergent composition, such composition further comprising an antimicrobial agent as disclosed hereinafter, preferably selected from the group consisting of 2-phenoxyethanol, more preferably comprising said antimicrobial agent in an amount ranging from 2 ppm to 5% by weight of the composition; even more preferably comprising 0.1 to 2% of phenoxyethanol.

In a further embodiment, this invention also encompasses a method of preserving an aqueous composition against microbial contamination or growth, such composition, specifically a cleaning composition, more preferably a cleaning composition in liquid, solid or semi-solid form, preferably being a concentrated liquid detergent formulation, single mono doses laundry detergent formulation, liquid hand dish washing detergent formulation or solid automatic dish washing formulation, more preferably a laundry detergent formulation, comprising an alkoxylated amino acid esters and/or their salts obtained by a process of the invention and in the amounts detailed before, such composition being preferably a detergent composition, such method comprising adding at least one antimicrobial agent selected from the disclosed antimicrobial agents as disclosed hereinafter, such antimicrobial agent preferably being 2-phenoxyethanol.

In a further embodiment, this invention also encompasses a method of laundering fabric or of cleaning hard surfaces, which method comprises treating a fabric or a hard surface with a cleaning composition, more preferably a liquid laundry detergent composition or a liquid hand dish composition, even more preferably a liquid laundry detergent composition, or a liquid softener composition for use in laundry, such composition comprising an alkoxylated amino acid esters and/or their salts obtained by a process of the invention in the amounts detailed before, such composition further comprising 4,4′-dichloro 2-hydroxydiphenylether.

As used herein the phrase “cleaning composition” as used for the inventive compositions and products includes compositions and formulations designed for cleaning soiled material. Such compositions include but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, dish washing compositions, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation. The cleaning compositions may have a form selected from liquid, powder, single-phase or multi-phase unit dose, pouch, tablet, gel, paste, bar, or flake.

The cleaning compositions of the invention comprise a surfactant system in an amount sufficient to provide desired cleaning properties. In some embodiments, the cleaning composition comprises, by weight of the composition, from about 1% to about 70% of a surfactant system. In other embodiments, the liquid cleaning composition comprises, by weight of the composition, from about 2% to about 60% of the surfactant system. In further embodiments, the cleaning composition comprises, by weight of the composition, from about 5% to about 30% of the surfactant system. In embodiments for for a liquid hand dishwashing or spray detergent cleaning composition such composition comprises preferably from 60% to 90%, more preferably from 70% to 80% by weight of the surfactant system, more preferably of an anionic surfactant. The surfactant system may comprise a detersive surfactant selected from anionic surfactants, nonionic surfactants, cationic surfactants, zwitterionic surfactants, amphoteric surfactants, ampholytic surfactants, and mixtures thereof. Those of ordinary skill in the art will understand that a detersive surfactant encompasses any surfactant or mixture of surfactants that provide cleaning, stain removing, or laundering benefit to soiled material.

Even more preferably, the compositions or products of the present invention as detailed herein before comprising at least one inventive alkoxylated amino acid esters and/or their salts obtained by a process of the invention and in the amounts as specified in the previous paragraph, optionally further comprising at least one surfactant or a surfactant system in amounts from about 1% to about 70% by weight of the composition or product, are preferably those for primary cleaning (i.e. removal of stains) and more preferably within laundry applications, and may additionally comprise at least one enzyme selected from lipases, hydrolases, amylases, proteases, cellulases, mannanases, hemicellulases, phospholipases, esterases, xylanases, DNases, dispersins, pectinases, oxidoreductases, cutinases, lactases and peroxidases, more preferably at least two of the aforementioned types.

The phrase “cleaning composition” as used herein includes compositions and formulations and products designed for cleaning soiled material. Such compositions, formulations and products include those designed for cleaning soiled material or soiled surfaces of any kind.

Compositions for “industrial and institutional cleaning” includes such cleaning compositions being designed for use in industrial and institutional cleaning, such as those for use of cleaning soiled material or surfaces of any kind, such as hard surface cleaners for surfaces of any kind, including tiles, carpets, PVC-surfaces, wooden surfaces, metal surfaces, lacquered surfaces.

“Compositions for Fabric and Home Care” include cleaning compositions including but not limited to laundry cleaning compositions and detergents, fabric softening compositions, fabric enhancing compositions, fabric freshening compositions, laundry prewash, laundry pretreat, laundry additives, spray products, dry cleaning agent or composition, laundry rinse additive, wash additive, post-rinse fabric treatment, ironing aid, dish washing compositions, hard surface cleaning compositions, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the rinse or wash cycle of the laundering operation, preferably during the wash cycle of the laundering or dish washing operation.

The cleaning compositions of the invention may be in any form, namely, in the form of a liquid; a solid such as a powder, granules, agglomerate, paste, tablet, pouches, bar, gel; an emulsion; types delivered in dual- or multi-compartment containers; single-phase or multi-phase unit dose; a spray or foam detergent; premoistened wipes (i.e., the cleaning composition in combination with a nonwoven material such as that discussed in U.S. Pat. No. 6,121,165, Mackey, et al.); dry wipes (i.e., the cleaning composition in combination with a nonwoven materials, such as that discussed in U.S. Pat. No. 5,980,931, Fowler, et al.) activated with water by a user or consumer; and other homogeneous, non-homogeneous or single-phase or multiphase cleaning product forms.

The liquid cleaning compositions of the present invention preferably have a viscosity of from 50 to 10000 mPa*s; liquid manual dish wash cleaning compositions (also liquid manual “dish wash compositions”) have a viscosity of preferably from 100 to 10000 mPa*s, more preferably from 200 to 5000 mPa*s and most preferably from 500 to 3000 mPa*s at 20 1/s and 20° C.; liquid laundry cleaning compositions have a viscosity of preferably from 50 to 3000 mPa*s, more preferably from 100 to 1500 mPa*s and most preferably from 200 to 1000 mPa*s at 20 1/s and 20° C.

The cleaning compositions and formulations of the invention may—and preferably do-contain adjunct cleaning additives (also abbreviated herein as “adjuncts”), such adjuncts being preferably in addition to a surfactant system as defined before.

Suitable adjunct cleaning additives include builders, cobuilders, structurants or thickeners, clay soil removal/anti-redeposition agents, polymeric soil release agents, dispersants such as polymeric dispersing agents, polymeric grease cleaning agents, solubilizing agents, chelating agents, enzymes, enzyme stabilizing systems, bleaching compounds, bleaching agents, bleach activators, bleach catalysts, brighteners, malodor control agents, pigments, dyes, opacifiers, hueing agents, dye transfer inhibiting agents, chelating agents, suds boosters, suds suppressors (antifoams), color speckles, silver care, anti-tarnish and/or anti-corrosion agents, alkalinity sources, pH adjusters, pH-buffer agents, hydrotropes, scrubbing particles, antibacterial agents, anti-oxidants, softeners, carriers, processing aids, pro-perfumes, and perfumes.

Liquid cleaning compositions additionally may comprise—and preferably do comprise at least one of—rheology control/modifying agents, emollients, humectants, skin rejuvenating actives, and solvents.

Solid compositions additionally may comprise- and preferably do comprise at least one of—fillers, bleaches, bleach activators and catalytic materials.

Suitable examples of such cleaning adjuncts and levels of use are found in WO 99/05242, U.S. Pat. Nos. 5,576,282, 6,306,812 B1 and 6,326,348 B1.

Those of ordinary skill in the art will understand that a detersive surfactant encompasses any surfactant or mixture of surfactants that provide cleaning, stain removing, or laundering benefit to soiled material.

Hence, the cleaning compositions of the invention such as fabric and home care products, and formulations for industrial and institutional cleaning, more specifically such as laundry and manual dish wash detergents, preferably additionally comprise a surfactant system and, more preferably, also further adjuncts, as the one described above.

The surfactant system may be composed from one surfactant or from a combination of surfactants selected from anionic surfactants, non-ionic surfactants, cationic surfactants, zwitterionic surfactants, amphoteric surfactants, and mixtures thereof. Those of ordinary skill in the art will understand that a surfactant system for detergents encompasses any surfactant or mixture of surfactants that provide cleaning, stain removing, or laundering benefit to soiled material.

The cleaning compositions of the invention typically comprise a surfactant system in an amount sufficient to provide desired cleaning properties.

The liquid cleaning compositions of the present invention may have any suitable pH-value. Preferably the pH of the composition is adjusted to between 4 and 14. More preferably the composition has a pH of from 6 to 13, even more preferably from 6 to 10, most preferably from 7 to 9. The pH of the composition can be adjusted using pH modifying ingredients known in the art and is measured as a 10% product concentration in demineralized water at 25° C. For example, NaOH may be used and the actual weight % of NaOH may be varied and trimmed up to the desired pH such as pH 8.0. In one embodiment of the present invention, a pH>7 is adjusted by using amines, preferably alkanolamines, more preferably triethanolamine.

The selection of the additional surfactants and further ingredients in these embodiments may be dependent upon the application and the desired benefit.

All such cleaning compositions, their ingredients including (adjunct) cleaning additives, their general compositions and more specific compositions are known, as for example illustrated in the publications 800542 and 800500 as published by Protegas, Liechtenstein, and also from WO 2022/136409 and WO 2022/136408, wherein in any of the before prior art documents the (amino) esteramines and their salts within the general compositions and also each individualized specific cleaning composition disclosed in the beforementioned publications may be replaced partially or completely by the aminoacid esters and/or their salts of this present invention. In those beforementioned documents, also various types of formulations for cleaning compositions are disclosed; all such composition types the general compositions and also each individualized specific cleaning composition—can be equally applied also to those cleaning compositions contemplated herein.

Hence, the present invention also encompasses any and all of such disclosed compositions of the before-mentioned prior art-disclosures but further comprising at least one of the inventive aminoacid ester and/or its salt in addition to or as a replacement for any already ins such prior art-composition contained esteramine or esteramine salt or any such compound, which can be replaced by such inventive aminoacid ester or salt-such replacements in principle known to a person of skill in the art or readily obvious in view of the present invention-, with the content of the inventive aminoacid ester and/or its salt being present in said formulations at a concentration of generally from 0.05 to 20 wt. %, preferably up to 10 wt. %, more preferably 0.1 to 5 weight %, even more preferably at a concentration of 0.5 to 2 weight %.

General Description of Cleaning Compositions, Formulations and their Ingredients

Cleaning compositions such as fabric and home care products and formulations for industrial and institutional cleaning, more specifically such as laundry and manual dish wash detergents, are known to a person skilled in the art. Any composition etc. known to a person skilled in the art, in connection with the respective use, can be employed within the context of the present invention by including at least one inventive compound, preferably at least one such inventive compound in amounts suitable for expressing a certain property within such a composition, especially when such a composition is used in its area of use.

### Cleaning Additives

The cleaning compositions and formulations of the invention may—and preferably do-contain adjunct cleaning additives (also abbreviated herein as “adjuncts”), such adjuncts being preferably in addition to a surfactant system as defined before.

Suitable adjunct cleaning additives include builders, cobuilders, structurants or thickeners, clay soil removal/anti-redeposition agents, polymeric soil release agents, dispersants such as polymeric dispersing agents, polymeric grease cleaning agents, solubilizing agents, chelating agents, enzymes, enzyme stabilizing systems, bleaching compounds, bleaching agents, bleach activators, bleach catalysts, brighteners, malodor control agents, pigments, dyes, opacifiers, hueing agents, dye transfer inhibiting agents, chelating agents, suds boosters, suds suppressors (antifoams), color speckles, silver care, anti-tarnish and/or anti-corrosion agents, alkalinity sources, pH adjusters, pH-buffer agents, hydrotropes, scrubbing particles, antibacterial agents, anti-oxidants, softeners, carriers, processing aids, pro-perfumes, and perfumes. All such adjuncts are detailed and exemplified further below in the following chapters.

Liquid cleaning compositions additionally may comprise—and preferably do comprise at least one of—rheology control/modifying agents, emollients, humectants, skin rejuvenating actives, and solvents. Solid compositions additionally may comprise- and preferably do comprise at least one of—fillers, bleaches, bleach activators and catalytic materials.

Suitable examples of such cleaning adjuncts and levels of use are found in WO 99/05242, U.S. Pat. Nos. 5,576,282, 6,306,812 B1 and 6,326,348 B1.

Those of ordinary skill in the art will understand that a detersive surfactant encompasses any surfactant or mixture of surfactants that provide cleaning, stain removing, or laundering benefit to soiled material.

Hence, the cleaning compositions of the invention such as fabric and home care products, and formulations for industrial and institutional cleaning, more specifically such as laundry and manual dish wash detergents, preferably additionally comprise a surfactant system and, more preferably, also further adjuncts, as the one described above and below in more detail.

The surfactant system may be composed from one surfactant or from a combination of surfactants selected from anionic surfactants, non-ionic surfactants, cationic surfactants, zwitterionic surfactants, amphoteric surfactants, and mixtures thereof. Those of ordinary skill in the art will understand that a surfactant system for detergents encompasses any surfactant or mixture of surfactants that provide cleaning, stain removing, or laundering benefit to soiled material.

The cleaning compositions of the invention preferably comprise a surfactant system in an amount sufficient to provide desired cleaning properties. The surfactant system may comprise a detersive surfactant selected from anionic surfactants, non-ionic surfactants, cationic surfactants, zwitterionic surfactants, amphoteric surfactants, and mixtures thereof.

### Laundry Compositions

“Laundry composition” may be any composition, formulation or product which is intended for use in laundry including laundry care, laundry cleaning etc.; hence this term will be used in the following denoting any composition, formulation or product.

In laundry compositions, anionic surfactants contribute usually by far the largest share of surfactants within such formulation. Hence, preferably, the inventive cleaning compositions for use in laundry comprise at least one anionic surfactant and optionally further surfactants selected from any of the surfactant classes described herein, preferably from non-ionic surfactants and/or amphoteric surfactants and/or zwitterionic surfactants and/or cationic surfactants.

Cleaning compositions may—and preferably do—also contain anionic surfactants—which may be employed also in combinations of more than one other surfactant.

Nonlimiting examples of anionic surfactants—which may be employed also in combinations of more than one surfactant—useful herein include C9-C20 linear alkylbenzenesulfonates (LAS), C10-C20 primary, branched chain and random alkyl sulfates (AS); C10-C18 secondary (2,3)alkyl sulfates; C10-C18 alkyl alkoxy sulfates (AExS) wherein x is from 1 to 30; C10-C18 alkyl alkoxy carboxylates comprising 1 to 5 ethoxy units; mid-chain branched alkyl sulfates as discussed in U.S. Pat. Nos. 6,020,303 and 6,060,443; mid-chain branched alkyl alkoxy sulfates as discussed in U.S. Pat. Nos. 6,008,181 and 6,020,303; modified alkylbenzene sulfonate (MLAS) as discussed in WO 99/05243, WO 99/05242 and WO 99/05244; methyl ester sulfonate (MES); and alpha-olefin sulfonate (AOS).

Preferred examples of suitable anionic surfactants are alkali metal and ammonium salts of C8-C12-alkyl sulfates, of C12-C18-fatty alcohol ether sulfates, of C12-C18-fatty alcohol polyether sulfates, of sulfuric acid half-esters of ethoxylated C4-C12-alkylphenols (ethoxylation: 3 to 50 mol of ethylene oxide/mol), of C12-C18-alkylsulfonic acids, of C12-C18 sulfo fatty acid alkyl esters, for example of C12-C18 sulfo fatty acid methyl esters, of C10-C18-alkylarylsulfonic acids, preferably of n-C10-C18-alkylbenzene sulfonic acids, of C10-C18 alkyl alkoxy carboxylates and of soaps such as for example C8-C24-carboxylic acids. Preference is given to the alkali metal salts of the aforementioned compounds, particularly preferably the sodium salts.

In one embodiment of the present invention, anionic surfactants are selected from n-C10-C18-alkylbenzene sulfonic acids and from fatty alcohol polyether sulfates, which, within the context of the present invention, are in particular sulfuric acid half-esters of ethoxylated C12-C18-alkanols (ethoxylation: 1 to 50 mol of ethylene oxide/mol), preferably of n-C12-C18-alkanols.

In one embodiment of the present invention, also alcohol polyether sulfates derived from branched (i.e., synthetic) C11-C18-alkanols (ethoxylation: 1 to 50 mol of ethylene oxide/mol) may be employed.

Preferably, the alkoxylation group of both types of alkoxylated alkyl sulfates, based on C12-C18-fatty alcohols or based on branched (i.e., synthetic) C11-C18-alcohols, is an ethoxylation group and an average ethoxylation degree of any of the alkoxylated alkyl sulfates is 1 to 5, preferably 1 to 3. Preferably, the laundry detergent formulation of the present invention comprises from at least 1 wt. % to 50 wt. %, preferably in the range from greater than or equal to about 2 wt. % to equal to or less than about 30 wt. %, more preferably in the range from greater than or equal to 3 wt. % to less than or equal to 25 wt. %, and most preferably in the range from greater than or equal to 5 wt. % to less than or equal to 25 wt. % of one or more anionic surfactants as described above, based on the particular overall composition, including other components and water and/or solvents.

In a preferred embodiment of the present invention, anionic surfactants are selected from C10-C15 linear alkylbenzenesulfonates, C10-C18 alkylethersulfates with 1-5 ethoxy units and C10-C18 alkylsulfates.

Cleaning compositions may also contain non-ionic surfactants—which may be employed also in combinations of more than one other surfactant.

Non-limiting examples of non-ionic surfactants—which may be employed also in combinations of more than one other surfactant-include: C8-C18 alkyl ethoxylates, such as, NEODOL® non-ionic surfactants from Shell; ethylenoxide/propylenoxide block alkoxylates as PLURONIC® from BASF; C14-C22 mid-chain branched alkyl alkoxylates, BAEx, wherein x is from 1 to 30, as discussed in U.S. Pat. Nos. 6,153,577, 6,020,303 and 6,093,856; alkylpolysaccharides as discussed in U.S. Pat. No. 4,565,647 Llenado, issued Jan. 26, 1986; specifically alkylpolyglycosides as discussed in U.S. Pat. Nos. 4,483,780 and 4,483,779; polyhydroxy fatty acid amides as discussed in U.S. Pat. No. 5,332,528; and ether capped poly(oxyalkylated) alcohol surfactants as discussed in U.S. Pat. No. 6,482,994 and WO 01/42408. Preferred examples of non-ionic surfactants are in particular alkoxylated alcohols and alkoxylated fatty alcohols, di- and multiblock copolymers of ethylene oxide and propylene oxide and reaction products of sorbitan with ethylene oxide or propylene oxide, furthermore alkylphenol ethoxylates, alkyl glycosides, polyhydroxy fatty acid amides (glucamides). Examples of (additional) amphoteric surfactants are so-called amine oxides.

Preferred examples of alkoxylated alcohols and alkoxylated fatty alcohols are, for example, compounds of the general formula (A)

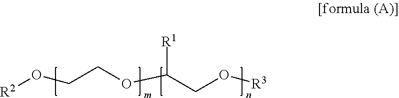

in which the variables are defined as follows:
R1 is selected from linear C1-C10-alkyl, preferably ethyl and particularly preferably methyl,
R2 is selected from C8-C22-alkyl, for example n-C8H17, n-C10H21, n-C12H25, n-C14H29, nC16H33 or n-C18H37,
R3 is selected from C1-C10-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl,
m and n are in the range from zero to 300, where the sum of n and m is at least one. Preferably, m is in the range from 1 to 100 and n is in the range from 0 to 30.

Here, compounds of the general formula (A) may be block copolymers or random copolymers, preference being given to block copolymers.

Other preferred examples of alkoxylated alcohols and alkoxylated fatty alcohols are, for example, compounds of the general formula (B)

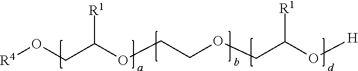

in which the variables are defined as follows:
R1 is identical or different and selected from linear C1-C4-alkyl, preferably identical in each case and ethyl and particularly preferably methyl,
R4 is selected from C6-C20-alkyl, in particular n-C8H17, n-C10H21, n-C12H25, n-C14H29, nC16H33, n-C18H37,
a is a number in the range from zero to 6, preferably 1 to 6,
b is a number in the range from zero to 20, preferably 4 to 20,
d is a number in the range from 4 to 25.

Preferably, at least one of a and b is greater than zero.

Here, compounds of the general formula (B) may be block copolymers or random copolymers, preference being given to block copolymers.

Further suitable non-ionic surfactants are selected from di- and multiblock copolymers, composed of ethylene oxide and propylene oxide. Further suitable non-ionic surfactants are selected from ethoxylated or propoxylated sorbitan esters. Alkylphenol ethoxylates or alkyl polyglycosides or polyhydroxy fatty acid amides (glucamides) are likewise suitable. An overview of suitable further non-ionic surfactants can be found in EP A 0 851 023 and in DE-A 198 19 187.

Mixtures of two or more different non-ionic surfactants may of course also be present.

In a preferred embodiment of the present invention, non-ionic surfactants are selected from C12/14 and C16/18 fatty alkoholalkoxylates, C13/15 oxoalkoholalkoxylates, C13-alkoholalkoxylates, and 2-propylheptylalkoholalkoxylates, each of them with 3-15 ethoxy units, preferably 5-10 ethoxy units, or with 1-3 propoxy- and 2-15 ethoxy units.

Cleaning compositions may also contain amphoteric surfactants—which may be employed also in combinations of more than one other surfactant.

Non-limiting examples of amphoteric surfactants—which may be employed also in combinations of more than one other surfactant-include: water-soluble amine oxides containing one alkyl moiety of from about 8 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms. See WO 01/32816, U.S. Pat. Nos. 4,681,704, and 4,133,779. Suitable surfactants include thus so-called amine oxides, such as lauryl dimethyl amine oxide (“lauramine oxide”). Preferred examples of amphoteric surfactants are amine oxides. Preferred amine oxides are alkyl dimethyl amine oxides or alkyl amido propyl dimethyl amine oxides, more preferably alkyl dimethyl amine oxides and especially coco dimethyl amino oxides. Amine oxides may have a linear or mid-branched alkyl moiety. Typical linear amine oxides include water-soluble amine oxides containing one R1=C8-18 alkyl moiety and two R2 and R3 moieties selected from the group consisting of C1-C3 alkyl groups and C1-C3 hydroxyalkyl groups. Preferably, the amine oxide is characterized by the formula

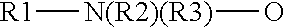

wherein R1 is a C8-18 alkyl and R2 and R3 are selected from the group consisting of methyl, ethyl, propyl, isopropyl, 2-hydroxethyl, 2-hydroxypropyl and 3-hydroxypropyl. The linear amine oxide surfactants in particular may include linear C10-C18 alkyl dimethyl amine oxides and linear C8-C12 alkoxy ethyl dihydroxy ethyl amine oxides. Preferred amine oxides include linear C10, linear C10-C12, and linear C12-C14 alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the alpha carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2) such that the one alkyl moiety and the one alkyl branch are symmetric. As used herein “symmetric” means that (n1-n2) is less than or equal to 5, preferably 4, most preferably from 0 to 4 carbon atoms in at least 50 wt. %, more preferably at least 75 wt. % to 100 wt. % of the mid-branched amine oxides for use herein. The amine oxide further comprises two moieties, independently selected from a C1-C3 alkyl, a C1-C3 hydroxyalkyl group, or a polyethylene oxide group containing an average of from about 1 to about 3 ethylene oxide groups. Preferably the two moieties are selected from a C1-C3 alkyl, more preferably both are selected as a C1 alkyl.

In a preferred embodiment of the present invention, amphoteric surfactants are selected from C8-C18 alkyl-dimethyl aminoxides and C8-C18 alkyl-di(hydroxyethyl)aminoxide.

Cleaning compositions may also contain zwitterionic surfactants—which may be employed also in combinations of more than one other surfactant.

Suitable zwitterionic surfactants include betaines, such as alkyl betaines, alkylamidobetaine, amidazoliniumbetaine, sulfobetaine (INCI Sultaines) as well as the phosphobetaines. Examples of suitable betaines and sulfobetaines are the following (designated in accordance with INCI): Almond amidopropyl of betaines, Apricotamidopropyl betaines, Avocadamidopropyl of betaines, Babassuamidopropyl of betaines, Behenamidopropyl betaines, Behenyl of betaines, Canol amidopropyl betaines, Capryl/Capramidopropyl betaines, Carnitine, Cetyl of betaines, Cocamidoethyl of betaines, Cocamidopropyl betaines, Cocamidopropyl Hydroxysultaine, Coco betaines, Coco Hydroxysultaine, Coco/Oleam idopropyl betaines, Coco Sultaine, Decyl of betaines, Dihydroxyethyl Oleyl Glycinate, Dihydroxyethyl Soy Glycinate, Dihydroxyethyl Stearyl Glycinate, Dihydroxyethyl Tallow Glycinate, Dimethicone Propyl of PG-betaines, Erucamidopropyl Hydroxysultaine, Hydrogenated Tallow of betaines, Isostearamid-opropyl betaines, Lauramidopropyl betaines, Lauryl of betaines, Lauryl Hydroxysultaine, Lauryl Sultaine, Milkamidopropyl betaines, Minkamidopropyl of betaines, Myristamidopropyl betaines, Myristyl of betaines, Oleamidopropyl betaines, Oleamidopropyl Hydroxysultaine, Oleyl of betaines, Olivamidopropyl of betaines, Palmamidopropyl betaines, Palmitamidopropyl betaines, Palmitoyl Carnitine, Palm Kernelamidopropyl betaines, Polytetrafluoroethylene Acetoxypropyl of betaines, Ricinoleam idopropyl betaines, Sesamidopropyl betaines, Soyamidopropyl betaines, Stearamidopropyl betaines, Stearyl of betaines, Tallowamidopropyl betaines, Tallowamidopropyl Hydroxysultaine, Tallow of betaines, Tallow Dihydroxyethyl of betaines, Undecylenamidopropyl betaines and Wheat Germamidopropyl betaines.

Preferred betaines are, for example, C12-C18-alkylbetaines and sulfobetaines. The zwitterionic surfactant preferably is a betaine surfactant, more preferable a Cocoamidopropylbetaine surfactant. Non-limiting examples of cationic surfactants—which may be employed also in combinations of more than one other surfactant-include: the quaternary ammonium surfactants, which can have up to 26 carbon atoms include: alkoxylated quaternary ammonium (AQA) surfactants as discussed in U.S. Pat. No. 6,136,769; dimethyl hydroxyethyl quaternary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Compositions according to the invention may comprise at least one builder. In the context of the present invention, no distinction will be made between builders and such components elsewhere called “co-builders”. Examples of builders are complexing agents, hereinafter also referred to as complexing agents, ion exchange compounds, and precipitating agents. Builders are selected from citrate, phosphates, silicates, carbonates, phosphonates, amino carboxylates and polycarboxylates. In the context of the present invention, the term citrate includes the mono- and the dialkali metal salts and in particular the mono- and preferably the trisodium salt of citric acid, ammonium or substituted ammonium salts of citric acid as well as citric acid. Citrate can be used as the anhydrous compound or as the hydrate, for example as sodium citrate dihydrate. Quantities of citrate are calculated referring to anhydrous trisodium citrate.

The term phosphate includes sodium metaphosphate, sodium orthophosphate, sodium hydrogenphosphate, sodium pyrophosphate and polyphosphates such as sodium tripolyphosphate. Preferably, however, the composition according to the invention is free from phosphates and polyphosphates, with hydrogenphosphates being subsumed, for example free from trisodium phosphate, pentasodium tripolyphosphate and hexasodium metaphosphate (“phosphate-free”). In connection with phosphates and polyphosphates, “free from” should be understood within the context of the present invention as meaning that the content of phosphate and polyphosphate is in total in the range from 10 ppm to 0.2% by weight of the respective composition, determined by gravimetry. The term carbonates includes alkali metal carbonates and alkali metal hydrogen carbonates, preferred are the sodium salts. Particularly preferred is Na2CO3.

Examples of phosphonates are hydroxyalkanephosphonates and aminoalkane¬phosphonates. Among the hydroxyalkanephosphonates, the 1-hydroxyethane-1,1-diphosphonate (HEDP) is of particular importance as builder. It is preferably used as sodium salt, the disodium salt being neutral and the tetrasodium salt being alkaline (pH 9). Suitable aminoalkanephosphonates are preferably ethylene diamine¬tetra¬methylene¬phosphonate (EDTMP), diethylenetriaminepenta¬methylene¬phosphonate (DTPMP), and also their higher homologues. They are preferably used in the form of the neutrally reacting sodium salts, e.g. as hexasodium salt of EDTMP or as hepta- and octa-sodium salts of DTPMP.

Examples of amino carboxylates and polycarboxylates are nitrilotriacetates, ethylene diamine tetraacetate, diethylene triamine pentaacetate, triethylene tetraamine hexaacetate, propylene diamines tetraacetic acid, ethanol-diglycines, methylglycine diacetate, and glutamine diacetate. The term amino carboxylates and polycarboxylates also include their respective non-substituted or substituted ammonium salts and the alkali metal salts such as the sodium salts, in particular of the respective fully neutralized compound.

Silicates in the context of the present invention include in particular sodium disilicate and sodium metasilicate, alumosilicates such as for example zeolites and sheet silicates, in particular those of the formula α-Na2Si2O5, β-Na2Si2O5, and δ-Na2Si2O5.

Compositions according to the invention may contain one or more builder selected from materials not being mentioned above. Examples of builders are α-hydroxypropionic acid and oxidized starch. In one embodiment of the present invention, builder is selected from polycarboxylates. The term “polycarboxylates” includes non-polymeric polycarboxylates such as succinic acid, C2-C16-alkyl disuccinates, C2-C16-alkenyl disuccinates, ethylene diamine N,N′-disuccinic acid, tartaric acid diacetate, alkali metal malonates, tartaric acid monoacetate, propanetricarboxylic acid, butanetetracarboxylic acid and cyclopentanetetracarboxylic acid.

Oligomeric or polymeric polycarboxylates are for example polyaspartic acid or in particular alkali metal salts of (meth)acrylic acid homopolymers or (meth)acrylic acid copolymers.

Suitable co-monomers are monoethylenically unsaturated dicarboxylic acids such as maleic acid, fumaric acid, maleic anhydride, itaconic acid and citraconic acid. A suitable polymer is in particular polyacrylic acid, which preferably has a weight-average molecular weight Mw in the range from 2000 to 40 000 g/mol, preferably 2000 to 10 000 g/mol, in particular 3000 to 8000 g/mol. Further suitable copolymeric polycarboxylates are in particular those of acrylic acid with methacrylic acid and of acrylic acid or methacrylic acid with maleic acid and/or fumaric acid.

It is also possible to use copolymers of at least one monomer from the group consisting of monoethylenically unsaturated C3-C10-mono- or C4-C10-dicarboxylic acids or anhydrides thereof, such as maleic acid, maleic anhydride, acrylic acid, methacrylic acid, fumaric acid, itaconic acid and citraconic acid, with at least one hydrophilically or hydrophobically modified co-monomer as listed below. Suitable hydrophobic co-monomers are, for example, isobutene, diisobutene, butene, pentene, hexene and styrene, olefins with ten or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C22-α-olefin, a mixture of C20-C24-α-olefins and polyisobutene having on average 12 to 100 carbon atoms per molecule.

Suitable hydrophilic co-monomers are monomers with sulfonate or phosphonate groups, and also non-ionic monomers with hydroxyl function or alkylene oxide groups. By way of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols here can comprise 3 to 50, in particular 5 to 40 and especially 10 to 30 alkylene oxide units per molecule.

Particularly preferred sulfonic-acid-group-containing monomers here are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy) propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Particularly preferred phosphonate-group-containing monomers are vinylphosphonic acid and its salts.

Moreover, amphoteric polymers can also be used as builders.

Compositions according to the invention can comprise, for example, in the range from in total 0.1 to 70% by weight, preferably 10 to 50% by weight, preferably up to 20% by weight, of builder(s), especially in the case of solid formulations. Liquid formulations according to the invention preferably comprise in the range of from 0.1 to 8% by weight of builder.

Formulations according to the invention can comprise one or more alkali carriers. Alkali carriers ensure, for example, a pH of at least 9 if an alkaline pH is desired. Of suitability are, for example, the alkali metal carbonates, the alkali metal hydrogen carbonates, and alkali metal metasilicates mentioned above, and, additionally, alkali metal hydroxides. A preferred alkali metal is in each case potassium, particular preference being given to sodium. In one embodiment of the present invention, a pH>7 is adjusted by using amines, preferably alkanolamines, more preferably triethanolamine.

In one embodiment of the present invention, the laundry formulation or composition according to the invention comprises additionally at least one enzyme.

Useful enzymes are, for example, one or more hydrolases selected from lipases, amylases, proteases, cellulases, hemicellulases, phospholipases, esterases, pectinases, lactases and peroxidases, and combinations of at least two of the foregoing types.

In one embodiment, the composition according to the present invention comprises additionally at least one enzyme.

Preferably, the at least one enzyme is a detergent enzyme.

In one embodiment, the enzyme is classified as an oxidoreductase (EC 1), a transferase (EC 2), a hydrolase (EC 3), a lyase (EC 4), an isomerase (EC 5), or a ligase (EC 6) (the EC-numbering is according to Enzyme Nomenclature, Recommendations (1992) of the Nomenclature Committee of the International Union of Biochemistry and Molecular Biology including its supplements published 1993-1999). Preferably, the enzyme is a hydrolase (EC 3).

In a preferred embodiment, the enzyme is selected from the group consisting of proteases, amylases, lipases, cellulases, mannanases, hemicellulases, phospholipases, esterases, pectinases, lactases, peroxidases, xylanases, cutinases, pectate lyases, keratinases, reductases, oxidases, phenoloxidases, lipoxygenases, ligninases, pullulanases, tannases, pentosanases, malanases, betaglucanases, arabinosidases, hyaluronidases, chondroitinases, laccases, nucleases, DNase, phosphodiesterases, phytases, carbohydrases, galactanases, xanthanases, xyloglucanases, oxidoreductase, perhydrolases, aminopeptidase, asparaginase, carbohydrase, carboxypeptidase, catalase, chitinase, cyclodextrin glycosyltransferase, alpha-galactosidase, beta-galactosidase, glucoamylase, alpha-glucosidase, beta-glucosidase, invertase, ribonuclease, transglutaminase, and dispersins, and combinations of at least two of the foregoing types. More preferably, the enzyme is selected from the group consisting of proteases, amylases, lipases, cellulases, mannanases, xylanases, DNases, dispersins, pectinases, oxidoreductases, and cutinases, and combinations of at least two of the foregoing types. Most preferably, the enzyme is a protease, preferably, a serine protease, more preferably, a subtilisin protease.

Such enzyme(s) can be incorporated into the composition at levels sufficient to provide an effective amount for achieving a beneficial effect, preferably for primary washing effects and/or secondary washing effects, like antigreying or antipilling effects (e.g., in case of cellulases). Preferably, the enzyme is present in the composition at levels from about 0.00001% to about 5%, preferably from about 0.00001% to about 2%, more preferably from about 0.0001% to about 1%, or even more preferably from about 0.001% to about 0.5% enzyme protein by weight of the composition.

Preferably, the enzyme-containing composition further comprises an enzyme stabilizing system. Preferably, the enzyme-containing composition described herein comprises from about 0.001% to about 10%, from about 0.005% to about 8%, or from about 0.01% to about 6%, by weight of the composition, of an enzyme stabilizing system. The enzyme stabilizing system can be any stabilizing system which is compatible with the enzyme.

Preferably, the enzyme stabilizing system comprises at least one compound selected from the group consisting of polyols (preferably, 1,3-propanediol, ethylene glycol, glycerol, 1,2-propanediol, or sorbitol), salts (preferably, CaCl2, MgCl2, or NaCl), short chain (preferably, C1-C6) carboxylic acids (preferably, formic acid, formate (preferably, sodium formate), acetic acid, acetate, or lactate), borate, boric acid, boronic acids (preferably, 4-formyl phenylboronic acid (4-FPBA)), peptide aldehydes, peptide acetals, and peptide aldehyde hydrosulfite adducts. Preferably, the enzyme stabilizing system comprises a combination of at least two of the compounds selected from the group consisting of salts, polyols, and short chain carboxylic acids and preferably one or more of the compounds selected from the group consisting of borate, boric acid, boronic acids (preferably, 4-formyl phenylboronic acid (4-FPBA)), peptide aldehydes, peptide acetals, and peptide aldehyde hydrosulfite adducts. In particular, if proteases are present in the composition, protease inhibitors may be added, preferably selected from borate, boric acid, boronic acids (preferably, 4-FPBA), peptide aldehydes (preferably, peptide aldehydes like Z-VAL-H or Z-GAY-H), peptide acetals, and peptide aldehyde hydrosulfite adducts. Compositions according to the invention may comprise one or more bleaching agent (bleaches).

Preferred bleaches are selected from sodium perborate, anhydrous or, for example, as the monohydrate or as the tetrahydrate or so-called dihydrate, sodium percarbonate, anhydrous or, for example, as the monohydrate, and sodium persulfate, where the term “persulfate” in each case includes the salt of the peracid H2SO5 and also the peroxodisulfate.

In this connection, the alkali metal salts can in each case also be alkali metal hydrogen carbonate, alkali metal hydrogen perborate and alkali metal hydrogen persulfate. However, the dialkali metal salts are preferred in each case.

Formulations according to the invention can comprise one or more bleach catalysts. Bleach catalysts can be selected from oxaziridinium-based bleach catalysts, bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper- and ruthenium-amine complexes can also be used as bleach catalysts.

Formulations according to the invention can comprise one or more bleach activators, for example tetraacetyl ethylene diamine, tetraacetylmethylene diamine, tetra-acetylglycoluril, tetraacetylhexylene diamine, acylated phenolsulfonates such as for example n-nonanoyl- or isononanoyloxybenzene sulfonates, N-methylmorpholinium-acetonitrile salts (“MMA salts”), trimethylammonium acetonitrile salts, N-acylimides such as, for example, N-nonanoylsuccinimide, 1,5-diacetyl-2,2-dioxohexahydro-1,3,5-triazine (“DADHT”) or nitrile quats (trimethylammonium acetonitrile salts).

Formulations according to the invention can comprise one or more corrosion inhibitors. In the present case, this is to be understood as including those compounds which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxy-hydroquinone, gallic acid, phloroglucinol or pyrogallol.

In one embodiment of the present invention, formulations according to the invention comprise in total in the range from 0.1 to 1.5% by weight of corrosion inhibitor.

Formulations according to the invention may also comprise further cleaning polymers and/or soil release polymers.

The additional cleaning polymers may include, without limitation, “multifunctional polyethylene imines” (for example BASF's Sokalan® HP20) and/or “multifunctional diamines” (for example BASF's Sokalan® HP96). Such multifunctional polyethylene imines are typically ethoxylated polyethylene imines with a weight-average molecular weight Mw in the range from 3000 to 250000, preferably 5000 to 200000, more preferably 8000 to 100000, more preferably 8000 to 50000, more preferably 10000 to 30000, and most preferably 10000 to 20000 g/mol. Suitable multifunctional polyethylene imines have 80 wt. % to 99 wt. %, preferably 85 wt. % to 99 wt. %, more preferably 90 wt. % to 98 wt. %, most preferably 93 wt. % to 97 wt. % or 94 wt. % to 96 wt. % ethylene oxide side chains, based on the total weight of the materials. Ethoxylated polyethylene imines are typically based on a polyethylene imine core and a polyethylene oxide shell. Suitable polyethylene imine core molecules are polyethylene imines with a weight-average molecular weight Mw in the range of 500 to 5000 g/mol. Preferably employed is a molecular weight from 500 to 1000 g/mol, even more preferred is a Mw of 600 to 800 g/mol. The ethoxylated polymer then has on average 5 to 50, preferably 10 to 35 and even more preferably 20 to 35 ethylene oxide (EO) units per NH-functional group.

Suitable multifunctional diamines are typically ethoxylated C2 to C12 alkylene diamines, preferably hexamethylene diamine, which are further quaternized and optionally sulfated. Typical multifunctional diamines have a weight-average molecular weight Mw in the range from 2000 to 10000, more preferably 3000 to 8000, and most preferably 4000 to 6000 g/mol. In a preferred embodiment of the invention, ethoxylated hexamethylene diamine, furthermore quaternized and sulfated, may be employed, which contains on average 10 to 50, preferably 15 to 40 and even more preferably 20 to 30 ethylene oxide (EO) groups per NH-functional group, and which preferably bears two cationic ammonium groups and two anionic sulfate groups.

In a preferred embodiment of the present invention, the cleaning compositions may contain at least one multifunctional polyethylene imine and/or at least one multifunctional diamine, and especially any one or more of Sokalan® HP20, Sokalan® HP96, and/or any of those as claimed in WO2021/254828, WO2022/136408A1, WO2022/136409A1, WO2021/165468, WO2023/021103, WO2023/021104, WO2023/021105 and WO2023/117494.

In a preferred embodiment of the present invention, the cleaning compositions may contain at least one multifunctional polyethylene imine and/or at least one multifunctional di- and/or oligoamine, specifically any of the claimed polymers from WO2021/254828, WO2022/136408A1, WO2022/136409A1, WO2021/165468, WO2023/021103, WO2023/021104, WO2023/021105 and/or WO2023/117494, to improve the cleaning the cleaning performance, such as preferably improve the stain removal ability, especially the primary detergency of particulate stains on polyester fabrics of laundry detergents. The multifunctional polyethylene imines or multifunctional diamines or mixtures thereof according to the descriptions above may be added to the laundry detergents and cleaning compositions in amounts of generally from 0.05 to 15 wt. %, preferably from 0.1 to 10 wt. % and more preferably from 0.25 to 5 wt. % and even as low as up to 2 wt. %, based on the particular overall composition, including other components and water and/or solvents.

Thus, one aspect of the present invention is a laundry detergent composition, in particular a liquid laundry detergent, comprising (i) at least one inventive compound and (ii) at least one compound selected from multifunctional polyethylene imines and multifunctional diamines and mixtures thereof. In one embodiment of the present invention, the ratio of the at least one inventive compound and (ii) the at least one compound selected from multifunctional polyethylene imines and multifunctional diamines and mixtures thereof, is from 10:1 to 1:10, preferably from 5:1 to 1:5 and more preferably from 3:1 to 1:3.

Cleaning compositions, fabric and home care products and specifically the laundry formulations comprising the inventive compound may also comprise at least one antimicrobial agent (named also “preservative”).

An antimicrobial agent is a chemical compound that kills microorganisms or inhibits their growth or reproduction. Microorganisms can be bacteria, yeasts or molds. A preservative is an antimicrobial agent which may be added to aqueous products and compositions to maintain the original performance, characteristics and integrity of the products and compositions by killing contaminating microorganisms or inhibiting their growth.

The composition/formulation may contain one or more antimicrobial agents and/or preservatives as listed in patent WO2021/115912 A1 (“Formulations comprising a hydrophobically modified polyethyleneimine and one or more enzymes”) on pages 35 to 39.

Especially of interest for the cleaning compositions and fabric and home care products and specifically in the laundry formulations are any of the following antimicrobial agents and/or preservatives: 4,4′-dichloro 2-hydroxydiphenyl ether (further names: 5-chloro-2-(4-chlorophenoxy) phenol, Diclosan, DCPP), Tinosan® HP 100 (commercial product of BASF SE containing 30% of the antimicrobial active 4,4′-dichloro 2-hydroxydiphenylether); 2-Phenoxyethanol (further names: Phenoxyethanol, Methylphenylglycol, Phenoxetyethanol, ethylene glycol phenyl ether, Ethylene glycol monophenyl ether, 2-(phenoxy) ethanol, 2-phenoxy-1-ethanol); 2-bromo-2-nitropropane-1,3-diol (further names: 2-bromo-2-nitro-1,3-propanediol, Bronopol); Glutaraldehyde (further names: 1-5-pentandial, pentane-1,5-dial, glutaral, glutar-dialdehyde); Glyoxal (further names: ethandial, oxylaldehyde, 1,2-ethandial); 2-butyl-benzo[d]isothiazol-3-one (“BBIT”); 2-methyl-2H-isothiazol-3-one (“MIT”); 2-octyl-2H-isothiazol-3-one (“OIT”); 5-Chloro-2-methyl-2H-isothiazol-3-one (“CIT” or “CMIT”); Mixture of 5-chloro-2-methyl-2H-isothiazol-3-one (“CMIT”) and 2-methyl-2H-isothiazol-3-one (“MIT”) (Mixture of CMIT/MIT); 1,2-benzisothiazol-3 (2H)-one (“BIT”); Hexa-2,4-dienoic acid (trivial name “sorbic acid”) and its salts, e.g., calcium sorb-ate, sodium sorbate; potassium (E,E)-hexa-2,4-dienoate (Potassium Sorbate); Lactic acid and its salts; L-(+)-lactic acid; especially sodium lactate; Benzoic acid and salts of benzoic acid, e.g., sodium benzoate, ammonium benzo-ate, calcium benzoate, magnesium benzoate, MEA-benzoate, potassium benzoate; Salicylic acid and its salts, e.g., calcium salicylate, magnesium salicylate, MEA salicylate, sodium salicylate, potassium salicylate, TEA salicylate; Benzalkonium chloride, benzalkonium bromide, benzalkonium saccharinate; Didecyldimethylammonium chloride (“DDAC”); N-(3-aminopropyl)-N-dodecylpropane-1,3-diamine (“Diamine”); Peracetic acid; Hydrogen peroxide.

At least one antimicrobial agent or preservative may be added to the inventive composition in a concentration of 0.001 to 10% relative to the total weight of the composition.

Preferably, the composition contains 2-phenoxyethanol in a concentration of 0.1 to 2% or 4,4′-dichloro 2-hydroxydiphenyl ether (DCPP) in a concentration of 0.005 to 0.6%.

The invention also encompasses a method of preserving an aqueous composition according to the invention against microbial contamination or growth, which method comprises addition of at least one antimicrobial agent or preservative, preferably 2-phenoxyethanol.

The invention also encompasses a method of providing an antimicrobial effect on textiles after treatment with a solid laundry detergent (e.g. powders, granulates, capsules, tablets, bars etc.), a liquid laundry detergent, a softener or an after-rinse containing 4,4′-dichloro 2-hydroxydiphenyl ether (DCPP).

Formulations according to the invention may also comprise water and/or additional organic solvents, e.g., ethanol or propylene glycol.

Further optional ingredients may be but are not limited to viscosity modifiers, cationic surfactants, foam boosting or foam reducing agents, perfumes, dyes, optical brighteners, and dye transfer inhibiting agents.

### Dish Wash Compositions

Another aspect of the present invention is also a dish wash composition, comprising at least one inventive compound(s) as described above.

Thus, an aspect of the present invention is also the use of the inventive compound(s) as described above, in dish wash applications, such as manual or automated dish wash applications.

Dish wash compositions according to the invention can be in the form of a liquid, semi-liquid, cream, lotion, gel, or solid composition, solid embodiments encompassing, for example, powders and tablets.

Liquid compositions are typically preferred for manual dish wash applications, whereas solid formulations and pouch formulations (where the pouches may contain also solids in addition to liquid ingredients) are typically preferred for automated dish washing compositions; however, in some areas of the world also liquid automated dish wash compositions are used and are thus of course also encompassed by the term “dish wash composition”.

The dish wash compositions are intended for direct or indirect application onto dishware and metal and glass surfaces, such as drinking and other glasses, beakers, dish and cooking ware like pots and pans, and cutlery such as forks, spoons, knives and the like.

The inventive method of cleaning dishware, metal and/or glass surfaces comprises the step of applying the dish wash cleaning composition, preferably in liquid form, onto the surface, either directly or by means of a cleaning implement, i.e., in neat form. The composition is applied directly onto the surface to be treated and/or onto a cleaning device or implement such as a dish cloth, a sponge or a dish brush and the like without undergoing major dilution (immediately) prior to the application. The cleaning device or implement is preferably wet before or after the composition is delivered to it. In the method of the invention, the composition can also be applied in diluted form.

Both neat and dilute application give rise to superior cleaning performance, i.e. the formulations of the invention containing at least one inventive compound(s) exhibit excellent degreasing properties. The effort of removing fat and/or oily soils from the dishware, metal and/or glass surfaces is decreased due to the presence of the inventive compound(s), even when the level of surfactant used is lower than in conventional compositions.

Preferably the composition is formulated to provide superior grease cleaning (degreasing) properties, long-lasting suds and/or improved viscosity control at decreased temperature exposures; preferably at least two, more preferably all three properties are present in the inventive dish wash composition. Optional—preferably present—further benefits of the inventive manual dish wash composition include soil removal, shine, and/or hand care; more preferably at least two and most preferably all three further benefits are present in the inventive dish wash composition.

In one embodiment of the present invention, the inventive compound(s) is one component of a manual dish wash formulation that additionally comprises at least one surfactant, preferably at least one anionic surfactant.

In another embodiment of the present invention, the inventive compound(s) is one component of a manual dish wash formulation that additionally comprises at least one anionic surfactant and at least one other surfactant, preferably selected from amphoteric surfactants and/or zwitterionic surfactants. In a preferred embodiment of the present invention, the manual dish wash formulations contain at least one amphoteric surfactant, preferably an amine oxide, or at least one zwitterionic surfactant, preferably a betaine, or mixtures thereof, to aid in the foaming, detergency, and/or mildness of the detergent composition.

Examples of suitable anionic surfactants are already mentioned above for laundry compositions. Preferred anionic surfactants for dish wash compositions are selected from C10-C15 linear alkylbenzenesulfonates, C10-C18 alkylethersulfates with 1-5 ethoxy units and C10-C18 alkylsulfates.

Preferably, the manual dish wash detergent formulation of the present invention comprises from at least 1 wt % to 50 wt %, preferably in the range from greater than or equal to about 3 wt % to equal to or less than about 35 wt %, more preferably in the range from greater than or equal to 5 wt % to less than or equal to 30 wt %, and most preferably in the range from greater than or equal to 5 wt % to less than or equal to 20 wt % of one or more anionic surfactants as described above, based on the particular overall composition, including other components and water and/or solvents.

Dish wash compositions according to the invention may comprise at least one amphoteric surfactant.

Examples of suitable amphoteric surfactants for dish wash compositions are already mentioned above for laundry compositions.

Preferred amphoteric surfactants for dish wash compositions are selected from C8-C18 alkyl-dimethyl aminoxides and C8-C18 alkyl-di(hydroxyethyl)aminoxide.

The manual dish wash detergent composition of the invention preferably comprises from 1 wt % to 15 wt %, preferably from 2 wt % to 12 wt %, more preferably from 3 wt % to 10 wt % of the composition of an amphoteric surfactant, preferably an amine oxide surfactant. Preferably the composition of the invention comprises a mixture of the anionic surfactants and alkyl dimethyl amine oxides in a weight ratio of less than about 10:1, more preferably less than about 8:1, more preferably from about 5:1 to about 2:1.

Addition of the amphoteric surfactant provides good foaming properties in the dish wash composition.

Dish wash compositions according to the invention may comprise at least one zwitterionic surfactant.

Examples of suitable zwitterionic surfactants for dish wash compositions are already mentioned above for laundry compositions.

Preferred zwitterionic surfactants for dish wash compositions are selected from betaine surfactants, more preferable from Cocoamidopropylbetaine surfactants.

In a preferred embodiment of the present invention, the zwitterionic surfactant is Cocamidopropylbetaine.

The manual dish wash detergent composition of the invention optionally comprises from 1 wt % to 15 wt %, preferably from 2 wt % to 12 wt %, more preferably from 3 wt % to 10 wt % of the composition of a zwitterionic surfactant, preferably a betaine surfactant.

Dish wash compositions according to the invention may comprise at least one cationic surfactant. Examples of suitable cationic surfactants for dish wash compositions are already mentioned above for laundry compositions.

Cationic surfactants, when present in the composition, are present in an effective amount, more preferably from 0.1 wt % to 5 wt %, preferably 0.2 wt % to 2 wt % of the composition.

Dish wash compositions according to the invention may comprise at least one non-ionic surfactant. Examples of suitable non-ionic surfactants for dish wash compositions are already mentioned above for laundry compositions.

Preferred non-ionic surfactants are the condensation products of Guerbet alcohols with from 2 to 18 moles, preferably 2 to 15, more preferably 5-12 of ethylene oxide per mole of alcohol. Other preferred non-ionic surfactants for use herein include fatty alcohol polyglycol ethers, alkylpolyglucosides and fatty acid glucamides.

The manual hand dish detergent composition of the present invention may comprise from 0.1 wt % to wt %, preferably from 0.3 wt % to 5 wt %, more preferably from 0.4 wt % to 2 wt % of the composition, of a linear or branched C10 alkoxylated non-ionic surfactant having an average degree of alkoxylation of from 2 to 6, preferably from 3 to 5. Preferably, the linear or branched C10 alkoxylated non-ionic surfactant is a branched C10 ethoxylated non-ionic surfactant having an average degree of ethoxylation of from 2 to 6, preferably of from 3 to 5. Preferably, the composition comprises from 60 wt % to 100 wt %, preferably from 80 wt % to 100 wt %, more preferably 100 wt % of the total linear or branched C10 alkoxylated non-ionic surfactant of the branched C10 ethoxylated non-ionic surfactant. The linear or branched C10 alkoxylated non-ionic surfactant preferably is a 2-propylheptyl ethoxylated non-ionic surfactant having an average degree of ethoxylation of from 3 to 5. A suitable 2-propylheptyl ethoxylated non-ionic surfactant having an average degree of ethoxylation of 4 is Lutensol® XP40, commercially available from BASF SE, Ludwigshafen, Germany. The use of a 2-propylheptyl ethoxylated non-ionic surfactant having an average degree of ethoxylation of from 3 to 5 leads to improved foam levels and long-lasting suds.

Thus, one aspect of the present invention is a manual dish wash detergent composition, in particular a liquid manual dish wash detergent composition, comprising (i) at least one inventive esteramine and/or its salt, and (ii) at least one further 2-propylheptyl ethoxylated non-ionic surfactant having an average degree of ethoxylation of from 3 to 5.

Dish wash compositions according to the invention may comprise at least one hydrotrope in an effective amount, to ensure the compatibility of the liquid manual dish wash detergent compositions with water.

Suitable hydrotropes for use herein include anionic hydrotropes, particularly sodium, potassium, and ammonium xylene sulfonate, sodium, potassium and ammonium toluene sulfonate, sodium, potassium, and ammonium cumene sulfonate, and mixtures thereof, and related compounds, as disclosed in U.S. Pat. No. 3,915,903.

The liquid manual dish wash detergent compositions of the present invention typically comprise from 0.1 wt % to 15 wt % of the total liquid detergent composition of a hydrotrope, or mixtures thereof, preferably from 1 wt % to 10 wt %, most preferably from 2 wt % to 5 wt % of the total liquid manual dish wash composition.

Dish wash compositions according to the invention may comprise at least one organic solvent. Examples of organic solvents are C4-C14 ethers and diethers, glycols, alkoxylated glycols, C6-C16 glycol ethers, alkoxylated aromatic alcohols, aromatic alcohols, aliphatic branched alcohols, alkoxylated aliphatic branched alcohols, alkoxylated linear C1-C5 alcohols, linear C1-C5 alcohols, amines, C8-C14 alkyl and cycloalkyl hydrocarbons and halohydrocarbons, and mixtures thereof.

When present, the liquid dish wash compositions will contain from 0.01 wt % to 20 wt %, preferably from 0.5 wt % to 15 wt %, more preferably from 1 wt % to 10 wt %, most preferably from 1 wt % to 5 wt % of the liquid detergent composition of a solvent. These solvents may be used in conjunction with an aqueous liquid carrier, such as water, or they may be used without any aqueous liquid carrier being present. At higher solvent systems, the absolute values of the viscosity may drop but there is a local maximum point in the viscosity profile.

The dish wash compositions herein may further comprise from 30 wt % to 90 wt % of an aqueous liquid carrier, comprising water, in which the other essential and optional ingredients are dissolved, dispersed or suspended. More preferably the compositions of the present invention comprise from 45 wt % to 85 wt %, even more preferably from 60 wt % to 80 wt % of the aqueous liquid carrier. The aqueous liquid carrier, however, may contain other materials which are liquid, or which dissolve in the liquid carrier, at room temperature (25° C.) and which may also serve some other function besides that of an inert filler.

Dish wash compositions according to the invention may comprise at least one electrolyte.

Suitable electrolytes are preferably selected from inorganic salts, even more preferably selected from monovalent salts, most preferably sodium chloride.

The liquid manual dish wash compositions according to the invention may comprise from 0.1 wt % to 5 wt %, preferably from 0.2 wt % to 2 wt % of the composition of an electrolyte.

Manual dish wash formulations comprising the inventive esteramine and/or salt(s) thereof may also comprise at least one antimicrobial agent.

Examples of suitable antimicrobial agents for dish wash compositions are already mentioned above for laundry compositions.

The antimicrobial agent may be added to the inventive hand dish wash composition in a concentration of 0.0001 wt % to 10 wt % relative to the total weight of composition. Preferably, the formulation contains 2-phenoxyethanol in a concentration of 0.01 wt % to 5 wt %, more preferably 0.1 wt % to 2 wt % and/or 4,4′-dichloro 2-hydroxydiphenyl ether in a concentration of 0.001 wt % to 1 wt %, more preferably 0.002 wt % to 0.6 wt % (in all cases relative to the total weight of the composition).

Further additional ingredients are such as but not limited to conditioning polymers, cleaning polymers, surface modifying polymers, soil flocculating polymers, rheology modifying polymers, enzymes, structurants, builders, chelating agents, cyclic diamines, emollients, humectants, skin rejuvenating actives, carboxylic acids, scrubbing particles, bleach and bleach activators, perfumes, malodor control agents, pigments, dyes, opacifiers, beads, pearlescent particles, microcapsules, antibacterial agents, pH adjusters including NaOH and alkanolamines such as mono-ethanolamines and buffering means.

### Hand Dishwashing Liquid Composition

The liquid detergent composition comprising the esteramine technology according to the invention may be a hand dishwashing or spray detergent composition. Preferably the liquid hand dishwashing or spray detergent composition comprises from between 0.1 and 50%, preferably between 1% and 30% by weight of the detergent composition comprising the esteramine technology according to the invention. Preferably the pH of the detergent composition of the invention, measured as a 10% product concentration in demineralized water at 20° C., is adjusted to between 3 and 14, more preferably between 4 and 13, more preferably between 6 and 12 and most preferably between 8 and 10. The composition of the present invention can be Newtonian or non-Newtonian, preferably Newtonian. Preferably, the composition has a viscosity of from 10 mPa·s to 10,000 mPa·s, preferably from 100 mPa·s to 5,000 mPa·s, more preferably from 300 mPa·s to 2,000 mPa·s, or most preferably from 500 mPa·s to 1,500 mPa·s, alternatively combinations thereof. The viscosity is measured at 20° C. with a Brookfield RT Viscometer using spindle 31 with the RPM of the viscometer adjusted to achieve a torque of between 40% and 60%. The viscosity of the Spray detergent is preferably ranging 0.5-100 mPa·s.

The liquid hand dishwashing or spray detergent cleaning composition comprises from 0.1% to 50%, preferably from 1% to 35%, more preferably from 3% to 30%, by weight of the total composition of a surfactant system. The surfactant system preferably comprises from 60% to 90%, more preferably from 70% to 80% by weight of the surfactant system of an anionic surfactant. Alkyl sulphated anionic surfactants are preferred, particularly those selected from the group consisting of: alkyl sulphate, alkyl alkoxy sulphate preferably alkyl ethoxy sulphate, and mixtures thereof. The alkyl sulphated anionic surfactant preferably has an average alkyl chain length of from 8 to 18, preferably from 10 to 14, more preferably from 12 to 14, most preferably from 12 to 13 carbon atoms. The alkyl sulphated anionic surfactant preferably has an average degree of alkoxylation preferably ethoxylation, of less than 5, preferably less than 3, more preferably from 0.5 to 2.0, most preferably from 0.5 to 0.9. The alkyl sulphate anionic surfactant preferably has a weight average degree of branching of more than 10%, preferably more than 20%, more preferably more than 30%, even more preferably between 30% and 60%, most preferably between 30% and 50%. Suitable counterions include alkali metal cation earth alkali metal cation, alkanol ammonium or ammonium or substituted ammonium, but preferably sodium. Suitable examples of commercially available alkyl sulphate anionic surfactants include, those derived from alcohols sold under the Neodol® brand-name by Shell, or the Lial®, Isalchem®, and Safol® brand-names by Sasol, or some of the natural alcohols produced by The Procter & Gamble Chemicals company.

The surfactant system preferably comprises from 0.1% to 20%, more preferably from 0.5% to 15% and especially from 2% to 10% by weight of the liquid hand dishwashing detergent composition of a co-surfactant. Preferred co-surfactants are selected from the group consisting of an amphoteric surfactant, a zwitterionic surfactant, and mixtures thereof. The anionic surfactant to the co-surfactant weight ratio can be from 1:1 to 8:1, preferably from 2:1 to 5:1, more preferably from 2.5:1 to 4:1. The co-surfactant is preferably an amphoteric surfactant, more preferably an amine oxide surfactant. Preferably, the amine oxide surfactant is selected from the group consisting of: alkyl dimethyl amine oxide, alkyl amido propyl dimethyl amine oxide, and mixtures thereof, most preferably C12-C14 alkyl dimethyl amine oxide. Suitable zwitterionic surfactants include betaine surfactants, preferably cocamidopropyl betaine.

The surfactant system of the composition of the present invention may comprises from 0.1% to 25%, preferably from 0.5% to 20%, more preferably from 1% to 10%, most preferably from 1.5% to 5%, by weight of the surfactant system, of a non-ionic surfactant. Suitable nonionic surfactants can be selected from the group consisting of: alkoxylated non-ionic surfactant, alkyl polyglucoside (“APG”) surfactant, and mixtures thereof. Suitable alkoxylated non-ionic surfactants can be linear or branched, primary or secondary alkyl alkoxylated preferably alkyl ethoxylated non-ionic surfactants comprising on average from 9 to 15, preferably from 10 to 14 carbon atoms in its alkyl chain and on average from 5 to 12, preferably from 6 to 10, most preferably from 7 to 8, units of ethylene oxide per mole of alcohol. Most preferably, the alkyl polyglucoside surfactant has an average alkyl carbon chain length between 10 and 16, preferably between 10 and 14, most preferably between 12 and 14, with an average degree of polymerization of between 0.5 and 2.5 preferably between 1 and 2, most preferably between 1.2 and 1.6. C8-C16 alkyl polyglucosides are commercially available from several suppliers (e.g., Simusol® surfactants from Seppic Corporation; and Glucopon® 600 CSUP, Glucopon® 650 EC, Glucopon® 600 CSUP/MB, and Glucopon® 650 EC/MB, from BASF Corporation).

The detergent composition herein may optionally comprise a number of other adjunct ingredients such as builders (e.g., preferably citrate), chelants (e.g., preferably GLDA), conditioning polymers, cleaning polymers including polyalkoxylated polyalkylene imines, surface modifying polymers, soil flocculating polymers, sudsing polymers including EO-PO-EO triblock copolymers, grease cleaning amines including cyclic polyamines, structurants, emollients, humectants, skin rejuvenating actives, enzymes, carboxylic acids, scrubbing particles, bleach and bleach activators, perfumes, malodor control agents, pigments, dyes, opacifiers, beads, pearlescent particles, microcapsules, organic solvents, inorganic cations such as alkaline earth metals such as Ca/Mg-ions, antibacterial agents, preservatives, viscosity adjusters (e.g., salt such as NaCl, and other mono-, di- and trivalent salts) and pH adjusters and buffering means (e.g. carboxylic acids such as citric acid, HCl, NaOH, KOH, alkanolamines, phosphoric and sulfonic acids, carbonates such as sodium carbonates, bicarbonates, sesquicarbonates, borates, silicates, phosphates, imidazole and alike).

### General Cleaning Compositions and Formulations for Laundry and Dish Wash

The disclosed liquid formulations in this chapter may and preferably do comprise 0 to 2% 2-phenoxyethanol, preferably about 1%, in addition to all other mentioned ingredients.

The disclosed liquid formulations in this chapter may and preferably do comprise 0-0.2% 4,4′-dichloro 2-hydroxydiphenylether, preferably about 0.15%, in addition to all other mentioned ingredients.

The bleach-free solid laundry compositions may comprise 0-0.2% 4,4′-dichloro 2-hydroxydiphenylethe, preferably about 0.15%, in addition to all other mentioned ingredients.

The disclosed formulations in this chapter may and preferably do comprise one or more enzymes selected from those disclosed herein above, more preferably a protease and/or an amylase, wherein even more preferably the protease is a protease with at least 90% sequence identity to SEQ ID NO: 22 of EP1921147B1 and having the amino acid substitution R101E (according to BPN′ numbering) and wherein the amylase is an amylase with at least 90% sequence identity to SEQ ID NO: 54 of WO2021032881A1, such enzyme(s) preferably being present in the formulations at levels from about 0.00001% to about 5%, preferably from about 0.00001% to about 2%, more preferably from about 0.0001% to about 1%, or even more preferably from about 0.001% to about 0.5% enzyme protein by weight of the composition.

The tables in this chapter show general cleaning compositions of certain types, which correspond to typical compositions correlating with typical washing conditions as typically employed in various regions and countries of the world. The at least one inventive compound may be added to such formulation(s) in suitable amounts as outlined herein.

When no inventive compound is added, a shown formulation is a “comparative formulation”; when the amount chosen is in the general range as disclosed herein and specifically within ranges disclosed herein as preferred amounts for the various ingredients and the inventive compound, the formulation is a formulation according to the invention. Ingredients (other than the inventive compound) listed with amounts including “zero %” in the mentioned range may be present but not necessarily have to be present, in both the inventive and the comparative formulations. Hence, each number encompassed by a given range is meant to be included in the formulations shown in this chapter, and all variations and permutations possible are likewise meant to be included.

In a preferred embodiment the inventive compound is used in a laundry detergent.

Liquid laundry detergents according to the present invention are preferably composed of:

| | |
|---|---|
| 0.1-5% | of at least one inventive compound |
| 1-50% | of surfactants |
| 0.1-40% | of builders, cobuilders and/or chelating agents |
| 0.1-50% | other adjuncts |

water to add up 100%.

Preferred liquid laundry detergents according to the present invention are composed of:

| | |
|---|---|
| 0.5-2% | of at least one inventive compound |
| 5-40% | of anionic surfactants selected from C10-C15- |
| | LAS and C10-C18 alkyl ethersulfates containing |
| | 1-5 ethoxy-units |
| 1.5-10% | of nonioic surfactants selected from C10-C18- |
| | alkyl ethoxylates containing 3-10 ethoxy-units |
| 2-20% | of soluble organic builders/cobuilders selected |
| | from C10-C18 fatty acids, di- and tricarboxylic |
| | acids, hydroxy-di- and hydroxytricaboxylic acids, |
| | aminopolycarboxylates and polycarboxylic acids |
| 0.05-5% | of an enzyme system containing at least one |
| | enzyme suitable for detergent use and preferably |
| | also an enzyme stabilizing system |
| 0.5-20% | of mono- or diols selected from ethanol, |
| | isopropanol, ethylenglycol, or propylenglyclol |
| 0.1-20% | other adjuncts |

water to add up to 100%.

Solid laundry detergents (like e.g. powders, granules or tablets) according to the present invention are preferably composed of:

| | |
|---|---|
| 0.1-5% | of at least one inventive compound |
| 1-50% | of surfactants |
| 0.1-90% | of builders, cobuilders and/or chelating agents |
| 0-50% | of fillers |
| 0-40% | of bleach actives |
| 0.1-30% | of other adjuncts and/or water |

wherein the sum of the ingredients adds up 100%.

Preferred solid laundry detergents according to the present invention are composed of:

| | |
|---|---|
| 0.5-2% | of at least one inventive compound |
| 5-30% | of anionic surfactants selected from C10-C15- LAS, |
| | C10-C18 alkylsulfates and C10-C18 alkyl |
| | ethersulfates containing 1-5 ethoxy-units |
| 1.5-7.5% | of non-ionic surfactants selected from C10-C18- |
| | alkyl ethoxylates containing 3-10 ethoxy-units |
| 20-80% | of inorganic builders and fillers selected from |
| | sodium carbonate, sodium bicarbonate, zeolites, |
| | soluble silicates, sodium sulfate |
| 0.5-15% | of cobuilders selected from C10-C18 fatty acids, |
| | di- and tricarboxylic acids, hydroxydi- and |
| | hydroxytricarboxylic acids, aminopolycarboxylates |
| | and polycarboxylic acids |
| 0.1-5% | of an enzyme system containing at least one enzyme |
| | suitable for detergent use and preferably also an |
| | enzyme stabilizing system |
| 0.5-30% | of bleach actives |
| 0.1-20% | other adjuncts |

water to ad up to 100%

In a preferred embodiment at least one esteramine and/or salt thereof according to the present invention is used in a manual dish wash detergent.

Liquid manual dish wash detergents according to the present invention are composed of:

| | |
|---|---|
| 1-50% | of at least one inventive esteramine and/or salt |
| | thereof |
| 1-90% | of surfactants |
| 0.1-50% | of other adjuncts |

water to add up 100%.
Preferred liquid manual dish wash detergents according
to the present invention are composed of:

| | |
|---|---|
| 3-30% | of at least one inventive esteramine and/or salt |
| | thereof |
| 1-35% | of a surfactant system: |
| | 60% to 90%, more preferably from 70% to 80% by |
| | weight of the surfactant system of an anionic |
| | surfactant; |
| | 0.5% to 15% - by weight of the surfactant system - |
| | of a co-surfactant, preferably selected from the |
| | group consisting of an amphoteric surfactant, |
| | a zwitterionic surfactant, and mixtures thereof; |
| | 1% to 10% - by weight of the surfactant system - of |
| | a non-ionic surfactant; |
| 0-5% | of an enzyme, preferably also including an enzyme |
| | stabilizing system; |
| 0.5-20% | of mono- or diols selected from ethanol, |
| | isopropanol, ethylene glycol, or propylene glycol; |
| 0.1-20% | other adjuncts; |

water to add up to 100%.

Alternative preferred liquid manual dish wash detergents according to the present invention are composed of:

| | |
|---|---|
| 3-35% | of at least one inventive esteramine and/or salt |
| | thereof |
| 5-80% | of anionic surfactants selected from C10-C15- LAS, |
| | C10-C18 alkyl ethersulfates containing 1-5 ethoxy- |
| | units, and C10-C18 alkylsulfate |
| 2-10% | of Cocamidopropylbetaine |
| 0-10% | of Lauramine oxide |
| 0-2% | of a non-ionic surfactant, preferably a C10- |
| | Guerbet alcohol alkoxylate |
| 0-5% | of an enzyme, preferably Amylase, and preferably |
| | also an enzyme stabilizing system |
| 0.5-20% | of mono- or diols selected from ethanol, |
| | isopropanol, ethylenglycol, or propylenglyclol |
| 0.1-20% | other adjuncts |

water to add up to 100%

It is clear that the total amount of all ingredients within the disclosed formulations have to add up to “100” percent by weight of the total formulation.

In the following tables:

$$“ Inventive Compound ( s ) ” = at least one esteramine and / or salt thereof as described in this present invention$$

General formula for laundry detergent compositions according to the invention:

| | Ranges of Ingredient |
|---|---|
| | in Liquid frame |
| | formulations |
| Ingredient | (numbers: wt. %) |
| Linear alkyl benzene sulphonic acid | 0 to 30 |
| Coco fatty acid | 1 to 12 |
| Fatty alcohol ether sulphate | 0 to 25 |
| NaOH or mono or triethanol amine | Add up to pH 7.5 to pH 9.0 |
| Alcohol ethoxylate | 3 to 10 |
| 1,2-Propylene glycol | 1 to 10 |
| Ethanol | 0 to 4 |
| Sodium citrate | 0 to 8 |
| water | Up to 100 |

Liquid laundry frame formulations according to the invention:

| active | | | | | | |
|---|---|---|---|---|---|---|
| (numbers: wt. % active) | F1 | F2 | F3 | F4 | F5 | F6 |
| alcohol ethoxylat 7EO | 5.40 | 10.80 | 12.40 | 7.30 | 1.60 | 7.60 |
| Coco fatty acid K12-18 | 2.40 | 3.10 | 3.20 | 3.20 | 3.50 | 6.40 |
| Fatty alcohol ether | 5.40 | 8.80 | 7.10 | 7.10 | 5.40 | 14.00 |
| sulphate | | | | | | |
| Linear alkyl benzene | 5.50 | 0.00 | 14.50 | 15.50 | 10.70 | 0.00 |
| sulphonic acid | | | | | | |
| 1,2 Propandiol | 6.00 | 3.50 | 8.70 | 8.70 | 1.10 | 7.80 |
| Triethanolamine | 0 | 0 | 0 | 0 | 0 | 0 |
| Monoethanolamine | 0 | 0 | 4.00 | 4.30 | 0.30 | 0 |
| NaOH | 2.20 | 1.10 | 0 | 0 | 0 | 1.00 |
| Glycerol | 0 | 0.80 | 3.00 | 2.80 | 0 | 0 |
| Ethanol | 2.00 | 0 | 0 | 0 | 0.38 | 0.39 |
| Na citrate | 3.00 | 2.80 | 3.40 | 2.10 | 7.40 | 5.40 |
| Inventive | 0.1-5 | 0.1-5 | 0.1-5 | 0.1-5 | 0.1-5 | 0.1-5 |
| Compound(s) (total) | | | | | | |
| polymers | 0-10 | 0-10 | 0-10 | 0-10 | 0-10 | 0-10 |
| At least one enzyme | 0-1 | 0-1 | 0-1 | 0-1 | 0-1 | 0-1 |
| (each) | | | | | | |
| water | to 100 | to 100 | to 100 | to 100 | to 100 | to 100 |

Liquid laundry frame formulations according to the invention-continued:

| active | | | | | | | | |
|---|---|---|---|---|---|---|---|---|
| (numbers: wt % active) | F7 | F8 | F9 | F10 | F11 | F12 | F13 | F14 |
| alcohol ethoxylat 7EO | 3.80 | 0.30 | 13.30 | 8.00 | 5.70 | 20.00 | 9.20 | 29.00 |
| Coco fatty acid K12-18 | 2.80 | 3.00 | 1.70 | 1.80 | 2.50 | 5.00 | 8.60 | 10.40 |
| Fatty alcohol ether | 2.80 | 4.50 | 3.90 | 4.10 | 0 | 10.00 | 22.20 | 0 |
| sulphate | | | | | | | | |
| Linear alkyl benzene | 6.30 | 5.43 | 11.45 | 5.90 | 10.10 | 10.00 | 28.00 | 27.00 |
| sulphonic acid | | | | | | | | |
| 1,2 Propandiol | 0.50 | 0 | 2.50 | 0.40 | 6.00 | 10.00 | 7.00 | 7.00 |
| Triethanolamine | 0 | 0 | 0 | 8.10 | 0 | 0 | 0 | 0 |
| Monoethanolamine | 0.40 | 1.80 | 0 | 0 | 0 | 0 | 8.00 | 7.00 |
| NaOH | 0 | 0 | 2.20 | 0 | 3.30 | 1.50 | 0 | 0 |
| Glycerol | 0 | 0.60 | 0.20 | 1.90 | 0 | 0 | 7.00 | 10.00 |
| Ethanol | 0 | 0 | 1.84 | 0 | 0 | 0 | 0 | 0 |
| Na citrate | 4.60 | 3.30 | 3.30 | 1.40 | 0 | 1.50 | 0 | 0 |
| Inventive | 0.5-2 | 0.5-2 | 0.5-2 | 0.5-2 | 0.5-2 | 0.5-2 | 0.5-2 | 0.5-2 |
| compound(s) (total) | | | | | | | | |
| polymers | 0-10 | 0-10 | 0-10 | 0-10 | 0-10 | 0-10 | 0-10 | 0-10 |
| At least one enzyme | 0-1 | 0-1 | 0-1 | 0-1 | 0-1 | 0-3 | 0-3 | 0-3 |
| (each) | | | | | | | | |
| water | to 100 | to 100 | to 100 | to 100 | to 100 | to 100 | to 100 | to 100 |

Laundry powder frame formulations according to the invention:

| Actives | | | | | | |
|---|---|---|---|---|---|---|
| (numbers: wt. % active) | Bleach free Powder | | | | | |
| Alcohol ethoxylate 7EO | 0.6 | 0 | 1 | 0 | 0 | 5.2 |
| Coco fatty acid K12-18 | 1.2 | 0 | 0 | 0 | 0 | 0 |
| Fatty alcohol ether sulphate | 1.5 | 0 | 0 | 0 | 0 | 6 |
| Linear alkyl benzene sulphonic | 12.1 | 11.2 | 13.6 | 21.9 | 18.7 | 12.7 |
| acid | | | | | | |
| Bleach activator | 0 | 0 | 0 | 0 | 0 | 0 |
| Percarbonate | 0 | 0 | 0 | 0 | 0 | 0 |
| AcetateNa | 0 | 0 | 0 | 0.1 | 0 | 0.1 |
| CitrateNa | 0 | 0 | 0 | 0 | 0 | 14 |
| Na Silicate | 27.9 | 5.8 | 6.6 | 2 | 15 | 20.3 |
| Na Carbonate | 17.2 | 35 | 37.3 | 30.1 | 37 | 1 |
| Na Phosphate | 0 | 0 | 0 | 14 | 0.3 | 0 |
| Na Hydrogencarbonate | 0.7 | 0.9 | 0.5 | 2.7 | 0.4 | 10.5 |
| Zeolite4A | 4.2 | 0.1 | 5.1 | 10.2 | 1.8 | 11.6 |
| HEDP | 0 | 0 | 0 | 0 | 0 | 0.13 |
| MGDA | 0 | 1.1 | 0 | 0 | 0 | 0 |
| at least one enzyme (each) | 0-1.5 | 0-1.5 | 0-1.5 | 0-1.5 | 0-1.5 | 0-1.5 |
| Na Sulfate | 30.8 | 1.3 | 33 | 11 | 22 | 3 |
| Na Chloride | 0.2 | 43 | 0.1 | 0 | 0.1 | 0.1 |
| optical brightener | 0.02 | 0 | | 0.1 | 0.06 | |
| Inventive Compound(s) (total) | 0.2-5 | 0.2-5 | 0.2-5 | 0.2-5 | 0.2-5 | 0.2-5 |
| polymers | 0-10 | 0-10 | 0-10 | 0-10 | 0-10 | 0-10 |

Laundry powder frame formulations according to the invention—continued:

| Actives | | | | | | |
|---|---|---|---|---|---|---|
| (numbers: wt. % active) | Bleach containing Powder | | | | | |
| Alcohol ethoxylate 7EO | 1.2 | 5 | 4 | 0.5 | 0.5 | 0 |
| Coco fatty acid K12-18 | 0 | 0 | 0 | 0.3 | 0 | 0.6 |
| Fatty alcohol ether sulphate | 0 | 3.9 | 4.4 | 1.6 | 0 | 0 |
| Linear alkyl benzene | 7.6 | 12.1 | 11.5 | 12.2 | 6.5 | 10.4 |
| sulphonic acid | | | | | | |
| Bleach activator | 0.2 | 9.5 | 9.5 | 0.5 | 0.8 | 2.2 |
| Percarbonate | 3.6 | 19.4 | 16.6 | 2.2 | 11.5 | 5.8 |
| AcetateNa | 0 | 6.7 | 7.1 | 0.3 | 1 | 0.7 |
| CitrateNa | 0 | 1.6 | 8.2 | 0.3 | 0.9 | 1.7 |
| Na Silicate | 3.6 | 11.3 | 16.4 | 10.2 | 9.1 | 16.5 |
| Na Carbonate | 21.6 | 8.7 | 1.4 | 8 | 22.9 | 14.8 |
| Na Phospahte | 0 | 0 | 0 | 0 | 0 | 0 |
| Na Hydrogencarbonate | 0.2 | 2.8 | 1.6 | 0.8 | 0.5 | 0.5 |
| Zeolite4A | 1.6 | 1.4 | 2.4 | 1.6 | 1.8 | 2.3 |
| HEDP | 0 | 0.27 | 0.16 | 0 | 0 | 0.17 |
| MGDA | 0 | 0 | 0 | 0 | 0 | 0 |
| At least one enzyme | 0-1.5 | 0-1.5 | 0-1.5 | 0-1.5 | 0-1.5 | 0-1.5 |
| (each) | | | | | | |
| Na Sulfate | 51 | 4 | 6 | 57 | 38 | 37 |
| Na Chloride | 1 | 1 | 0.5 | 1.2 | 0.2 | 1 |
| optical brightener | | 0.29 | 0.1 | 0.23 | 0.13 | 0.19 |
| Inventive Compound(s) | 0.5-2 | 0.5-2 | 0.5-2 | 0.5-2 | 0.5-2 | 0.5-2 |
| (total) | | | | | | |
| polymers | 0-10 | 0-10 | 0-10 | 0-10 | 0-10 | 0-10 |

Further typical liquid detergent formulations LD1, LD2 and LD3 are shown in the following three tables: (numbers: wt. % active)

Liquid detergent 1—LD1 “excellent” detergent;

| Liquid Detergent Formulation | |
|---|---|
| Sodium alkylbenzene sulfonic acid (C₁₀-C₁₃) LAS | 9.5 |
| C₁₃/C₁₅-Oxoalkohol reacted with 7 moles of EO | 4.5 |
| 1,2 propyleneglycol | 6 |
| ethanol | 2 |
| potassium coconut soap | 2.4 |
| NaOH | 2.2 |
| lauryl ether sulphate (Texapon) | 5.0 |
| Sodium citrate | 3 |
| Sokalan HP 20 | 2 |
| Inventive Compound or comparison | 0.5-5 |
| Graft polymer* | 0-2 |
| Water | to 100 |

Liquid detergent 2—LD2 “medium” performance detergent

| Liquid Detergent Formulation | |
|---|---|
| Sodium alkylbenzene sulfonic acid (C₁₀-C₁₃) | 5.5 |
| C₁₃/C₁₅-Oxoalkohol reacted with 7 moles of EO | 5.4 |
| 1,2 propyleneglycol | 6 |
| ethanol | 2 |
| potassium coconut soap | 2.4 |
| Monoethanolamine | 2.5 |
| lauryl ether sulphate | 5.4 |
| Sodium citrate | 3 |
| Sokalan HP96 | 2 |
| Inventive Compound or comparison | 0.1-4 |
| Graft polymer* | 0-2 |
| Water | to 100 |

Liquid detergent 3—LD3 “medium” performance “biobased” detergent

| Liquid Detergent Formulation | |
|---|---|
| MGDA | 5.5 |
| APG, branched C13 Glucosid | 3.5 |
| 1,2 propyleneglycol | 6 |
| ethanol | 2 |
| potassium coconut soap | 4.4 |
| NaOH | 2.2 |
| lauryl ether sulphate | 9.5 |
| Sodium citrate | 3 |
| Inventive Compound or comparison | 0.1-4 |
| Graft polymer* | 0-2 |
| Water | to 100 |

All previous three tables on LD1, LD2, LD3: * “graft polymer”=(poly ethylene glycol of Mn 6000 g/mol as graft base, grafted with 40 weight % vinyl acetate (based on total polymer weight; produced following general disclosure of WO2007138054A1)

Liquid manual dish wash frame formulations according to the invention:

| Ingredients | MDW.1 | MDW.2 | MDW.3 | MDW.4 | MDW.5 | MDW.6 | MDW.7 | MDW.8 |
|---|---|---|---|---|---|---|---|---|
| Linear C₁₂-C₁₄-alkyl- | 8 | 0 | 6 | 0 | 6 | 0 | 6 | 0 |
| benzenesulfonic | | | | | | | | |
| acid | | | | | | | | |
| C₁₂-C₁₄-fatty | 8 | 16 | 6 | 12 | 6 | 12 | 6 | 12 |
| alcohol × 2 EO sulfate | | | | | | | | |
| Cocamidopropyl | 0 | 0 | 4 | 4 | 0 | 0 | 2 | 2 |
| betaine | | | | | | | | |
| Lauramine oxide | 0 | 0 | 0 | 0 | 4 | 4 | 2 | 2 |
| 2-Propylheptanol × | 0.5 | 0.5 | 0.5 | 0.5 | 0.5 | 0.5 | 0.5 | 0.5 |
| 4 EO | | | | | | | | |
| Inventive | 0.5-5 | 0.5-5 | 0.5-5 | 0.5-5 | 0.5-5 | 0.5-5 | 0.5-5 | 0.5-5 |
| Compound(s) (total) | | | | | | | | |
| Ethanol | 2 | 2 | 2 | 2 | 2 | 2 | 2 | 2 |
| Polymer(s) | 0-5 | 0-5 | 0-5 | 0-5 | 0-5 | 0-5 | 0-5 | 0-5 |
| 2-Phenoxyethanol | 1 | 1 | 1 | 1 | 1 | 1 | 1 | 1 |
| (preservative) | | | | | | | | |
| Sodium chloride | 1 | 1 | 1 | 1 | 1 | 1 | 1 | 1 |
| Demin. water | add 100 | add 100 | add 100 | add 100 | add 100 | add 100 | add 100 | add 100 |
| Sodium hydroxide | add | add | add | add | add | add | add | add |
| | pH 8 | pH 8 | pH 8 | pH 8 | pH 8 | pH 8 | pH 8 | pH 8 |

It is preferred, that within the respective laundry detergent, cleaning composition and/or fabric and home care product, the at least one esteramine and/or salt thereof as described in this invention is present at a concentration of from about 0.1% to about 10%, preferably from about 0.2% to 5%, more preferably from about 0.5% to about 5%, all in relation to the total weight of such composition or product in relation to the total weight of such composition or product, and all numbers in between, and including all ranges resulting from selecting any of the lower limits mentioned and including further 0.2, 0.3, 0.4, 1, 1.5, 2, 2.5, 3, 3.5 and 4, and combing with any of the upper limits mentioned and including 19, 18, 17, 16, 14, 13, 12, 11, 9, 8, 7, and 6.

Specifically for a liquid hand dishwashing or spray detergent composition the at least one esteramine and/or salt thereof as described in this invention is present at a concentration of from about between 0.1 and 50%, preferably between 1% and 30%, by weight of the detergent composition.

The specific embodiments as described throughout this disclosure are encompassed by the present invention as part of this invention; the various further options being disclosed in this present specification as “optional”, “preferred”, “more preferred”, “even more preferred” or “most preferred” options of a specific embodiment may be individually and independently (unless such independent selection is not possible by virtue of the nature of that feature or if such independent selection is explicitly excluded) selected and then combined within any of the other embodiments (where other such options and preferences can be also selected individually and independently), with each and any and all such possible combinations being included as part of this invention as individual embodiments, and especially with the preferred embodiments disclosed in the following section.

### EXAMPLES

### Methods

1H NMR measured in MeOD with Bruker Avance 400 MHz spectrometer.

From the NMR-data and specifically the mentioned peaks/range the respective conversion rate can be calculated using known means.

Hydroxy values are determined according to DIN 53240-1 as of 2016.

| Example | alkoxylated alcohol − aminoacid + sulfuric acid |
|---|---|
| 1 | Lutensol XP30 + L-Alanine + sulfuric acid |
| 2 | Lutensol XP30 + Valine + sulfuric acid |
| 3 | Dehydol LS 2 + Valine + sulfuric acid |
| 4 | Dehydol LS 2 + L-Alanine + sulfuric acid |
| 5 | Phenoxyethanol + Valine + sulfuric acid |

Dehydol LS 2 = C12-C14-alcohol alkoxylated with (on average) 2 EO/OH
Lutensol XP30 = 2-Propylheptanol, alkoxylated with (on average) 3 EO/OH

### Example 1: L-alanine-triethylene glycol-2-propylheptylether ester as triethylene glycol-2-propylheptylether-sulfuric acid salt

In a 4-neck vessel with thermometer, reflux condenser, nitrogen inlet, dropping funnel, and stirrer, 152.5 g 2-propylheptanol, ethoxylated with 3 mole ethylene oxide (Lutensol® XP30), 0.4 g hypophosphorous acid (50% in water), 22.3 g L-alanine and 4.5 g water were placed. 26.1 g concentrated sulfuric acid were added within 15 minutes. The temperature raised from room temperature to 59° C. during the addition. Reflux condenser was replaced by a distillation head, and the reaction mixture was heated to 135° C. bath temperature and stirred for 20 hours under a constant stream of nitrogen bubbling through the reaction mixture. Water was distilled off from the reaction mixture. To complete the reaction, vacuum (10 mbar) was applied, and the mixture was stirred 8 h under vacuum and 135° C. 175.0 g of a brown oil was obtained. 1H-NMR in MeOD indicated complete conversion to L-alanine-triethylene glycol-2-propylheptylether ester as triethylene glycol-2-propylheptylether-sulfuric acid salt. At δ=3.9-4.5 ppm a broad multiplet for esterified and sulfatized CH2-OH groups and proton at C2 from alanine was identified.

### Example 2: L-valine-triethylene glycol-2-propylheptylether ester as triethylene glycol-2-propylheptylether-sulfuric acid salt

In a 4-neck vessel with thermometer, reflux condenser, nitrogen inlet, dropping funnel, and stirrer, 152.5 g 2-propylheptanol, ethoxylated with 3 mole ethylene oxide (Lutensol® XP30), 0.4 g hypophosphorous acid (50% in water), 29.8 g L-valine and 4.0 g water were placed. 26.1 g concentrated sulfuric acid were added within 15 minutes. The temperature raised from room temperature to 55° C. during the addition. Reflux condenser was replaced by a distillation head, and the reaction mixture was heated to 135° C. bath temperature and stirred for 23 hours under a constant stream of nitrogen bubbling through the reaction mixture. Water was distilled off from the reaction mixture. 160.0 g of a brown oil was obtained. 1H-NMR in MeOD indicated 94% conversion to L-valine-triethylene glycol- 2-propylheptylether ester as triethylene glycol-2-propylheptylether-sulfuric acid salt. At δ=3.9-4.5 ppm a broad multiplet for esterified and sulfatized CH2-OH groups was identified.

### Example 3: L-valine-diethylene glycol-C12/C14-alkylester as diethylene glycol-C12/C14-alkylsulfuric acid salt

In a 4-neck vessel with thermometer, reflux condenser, nitrogen inlet, dropping funnel, and stirrer, 151.4 g C12/C14 fatty alcohol, ethoxylated with 2 mole ethylene oxide (hydroxy value 194 mgKOH/g), 0.4 g hypophosphorous acid (50% in water), 29.3 g L-valine and 4.5 g water were placed. 26.1 g concentrated sulfuric acid were added within 15 minutes. The temperature raised from room temperature to 43° C. during the addition. Reflux condenser was replaced by a distillation head, and the reaction mixture was heated to 135° C. bath temperature and stirred for 8 hours under a constant stream of nitrogen bubbling through the reaction mixture. Water was distilled off from the reaction mixture. Vacuum (10 mbar) was applied, and the reaction mixture was stirred for additional 4 hours under vacuum to complete the conversion. 164.7 g of a brown oil was obtained. 1H-NMR in MeOD indicated 94% conversion to L-valine-diethylene glycol-C12/C14-alkylester as diethylene glycol-C12/C14-alkylsulfuric acid salt. At δ=3.9-4.5 ppm a broad multiplet for esterified and sulfatized CH2-OH groups was identified.

### Example 4: L-alanine-diethylene glycol-C12/C14-alkylester as diethylene glycol-C12/C14-alkylsulfuric acid salt

In a 4-neck vessel with thermometer, reflux condenser, nitrogen inlet, dropping funnel, and stirrer, 151.4 g C12/C14 fatty alcohol, ethoxylated with 2 mole ethylene oxide (hydroxy value 194 mgKOH/g), 0.4 g hypophosphorous acid (50% in water), 22.3 g L-alanine and 4.5 g water were placed. 26.1 g concentrated sulfuric acid were added within 15 minutes. The temperature raised from room temperature to 45° C. during the addition. Reflux condenser was replaced by a distillation head, and the reaction mixture was heated to 135° C. bath temperature and stirred for 11 hours under a constant stream of nitrogen bubbling through the reaction mixture. Water was distilled off from the reaction mixture. 162.0 g of a brown oil was obtained. 1H-NMR in MeOD indicated 93% conversion to L-alanine-diethylene glycol-C12/C14-alkylester as diethylene glycol-C12/C14-alkylsulfuric acid salt. At δ=3.9-4.5 ppm a broad multiplet for esterified and sulfatized CH2-OH groups and proton at C2 from alanine was identified.

### Example 5: β-alanine-diethylene glycol-C12/C14-alkylester as diethylene glycol-C12/C14-alkylsulfuric acid salt

In a 4-neck vessel with thermometer, reflux condenser, nitrogen inlet, dropping funnel, and stirrer, 95.5 g C12/C14 fatty alcohol, ethoxylated with 2 mole ethylene oxide (hydroxy value 194 mgKOH/g), 0.25 g hypophosphorous acid (50% in water), 14.1 g β-alanine and 2.8 g water were placed. 16.5 g concentrated sulfuric acid were added within 15 minutes. The temperature raised from room temperature to 37° C. during the addition. Reflux condenser was replaced by a distillation head, and the reaction mixture was heated to 135° C. bath temperature and stirred for 8 hours under a constant stream of nitrogen bubbling through the reaction mixture. Water was distilled off from the reaction mixture. 100.8 g of a brown solid was obtained. 1H-NMR in MeOD indicated 96% conversion to β-alanine-diethylene glycol-C12/C14-alkylester as diethylene glycol-C12/C14-alkylsulfuric acid salt. At δ=3.9-4.4 ppm a broad multiplet for esterified and sulfatized CH2-OH groups was identified.

### Example 6: L-valine-phenoxyethanol ester as phenoxyethanol-sulfuric acid salt

In a 4-neck vessel with thermometer, reflux condenser, nitrogen inlet, dropping funnel, and stirrer, 145.1 g phenoxyethanol, 0.5 g hypophosphorous acid (50% in water), 58.6 g L-valine and 9.0 g water were placed. 52.1 g concentrated sulfuric acid were added within 15 minutes. The temperature raised from room temperature to 60° C. during the addition. Reflux condenser was replaced by a distillation head, and the reaction mixture was heated to 135° C. bath temperature and stirred for 6 hours under a constant stream of nitrogen bubbling through the reaction mixture. Water was distilled off from the reaction mixture. To complete the reaction, vacuum (10 mbar) was applied, and the mixture was stirred 6 h under vacuum and 135° C. 220.0 g of a yellow oil was obtained. 1H-NMR in MeOD indicated 89% conversion to L-valine-phenoxyethanol ester as phenoxyethanol-sulfuric acid salt. At δ=4.4-4.7 ppm two multiplets for esterified CH2-OH groups were identified, δ=4.1-4.3 ppm a multiplet for sulfatized CH2-OH groups and phenyl-O—CH2- was detected.

The following is an exemplary liquid hand dishwashing detergent formulation where the technology in scope is further included. The formulation can be made through standard mixing of the individual components according to known procedures.

| | Inventive Composition |
|---|---|
| Ingredients (As 100% active) | (in percent) |
| C1213 AE_{0.6}S anionic surfactant | 19.6 |
| (Average branching: 37.84%) | |
| C1214 dimethyl amine oxide | 6.5 |
| Alcohol ethoxylate nonionic | 1.0 |
| surfactant (Neodol 91/8) | |
| Alkoxylated polyethyleneimine | 0.2 |
| (PEI₆₀₀EO₂₄PO₁₆) | |
| Ethanol | 2.4 |
| NaCl | 0.7 |
| Polypropylene glycol | 0.9 |
| (Mol weight MW 2000 g/mol) | |
| Esteramine of the invention | 0.05-5 |
| Water + Minor ingredients | Balance to 100 |
| (perfume, dye, preservatives) | |
| pH (at 10% product concentration | 9.0 |
| in demineralized water - with | |
| NaOH trimming) | |

1.-18. (canceled)

19. A process for the production of an organoethersulfate salt of an alkoxylated amino acid ester (“AES”) comprising the steps of
(i) reacting at least one alpha-amino acid and at least one alkoxylated alcohol with sulfuric acid in an aqueous solution; and
(ii) optionally removing water or excess alcohol.

20. The process of claim 19, further comprising the steps of
(A) providing at least one alcohol bearing one hydroxy group, wherein such hydroxy group is alkoxylated with at least one alkylene oxide moiety;
(B) providing at least one alpha-amino acid;
(C) providing sulfuric acid; and
wherein the molar ratios are at least as follows:
the ratio between amino acid to hydroxyl groups of the alkoxylated alcohol is minimum 1:2 or 1: [more than 2];
the ratio of sulfuric acid to amino acid is 0.9:1 to 1.1:1;
the ratio between hydroxyl groups of alkoxylated alcohol and sulfuric acid is minimum 2:1; and
the molar ratio of water to amino acid is from 0.1:1 to 10:1;
(D) reacting the at least one alkoxylated alcohol with the at least one alpha-aminoacid in the presence of sulfuric acid to obtain the AES;
(E) optionally removing water or excess alcohol during and optionally also after the reaction to obtain the purified AES; and
(F) optionally neutralizing and purifying the AES to obtain the alkoxylated amino acid esters (“AAE”).

21. The process according to claim 19, wherein at least one of the following process steps is performed:
i) inertizing the reaction mixture before or during the reaction using a stream of gas such as nitrogen or argon, or by adding an oxygen-scavenging compound, such as at least one hypophosporous compound; and
ii) removing water or alkoxylated alcohol during and optionally also after the reaction, by applying a stream of gas or applying a distillation.

22. The process according to claim 19, wherein the reaction is performed
at a temperature of from 50 to 200° C.;
for a period of from 1 to 30; and
at from 0.001 to 10 bar pressure.

23. The process according to claim 19, wherein the at least one alkoxylated alcohol is selected from alkoxylated mono-alcohols such as C1- to C36-alkanols.

24. The process according to claim 19, wherein the at least one amino acid is selected from any alpha-amino acid such as alanine, glycine, leucine, isoleucine, valine, proline, phenylalanine, arginine, asparagine, aspartic acid, aspartate, glutamine, glutamate, histidine, lysine, threonine, tryptophan, tyrosine, cysteine, methionine, serine; alpha-amino acids with secondary or tertiary amino groups such as sarcosine, N,N-dimethylglycine.

25. The process according to claim 19, wherein the at least one alkoxylated alcohol comprises at least one alkylene oxide selected from C2 to C22-alkylene oxides.

26. The process according to claim 25, wherein the alkoxylated alcohol comprises at least one alkylene oxide being ethylene oxide or propylene oxide.

27. The process according to claim 19, wherein the alkylene oxide employed comprises at least 10% based on non-fossil derived carbon atoms.

28. The process according to claim 19, wherein no further organic solvent is introduced into the reaction as such besides the organic solvents possibly being present within any of the starting materials employed, i.e. within the sulfuric acid, the aminoacid(s) and the alkoxylated alcohol(s).

29. An alkoxylated amino acid ester (“AAE”) or its organoethersulfate salt (“AES”) obtained by the process according to claim 19.

30. A cleaning composition, fabric and home care product, industrial and institutional cleaning product, cosmetic or personal care product comprising at least one alkoxylated amino acid ester or at least one organoethersulfate salt according to claim 29.

31. The cleaning composition, fabric and home care product, industrial and institutional cleaning product, cosmetic or personal care product according to claim 30 for use in a fabric detergent formulation or dish wash detergent formulation.

32. A composition comprising at least one AAE or at least one AES obtained by the process according to claim 19.

33. The composition according to claim 32 being a cleaning composition, fabric and home care product, industrial and institutional cleaning product, cosmetic or personal care product, wherein the at least one AAE or at least one AES is present in the composition or product at a concentration of from about 0.1% to about 50% in weight % in relation to the total weight of such composition/product, optionally further comprising from about 1% to about 70% by weight of a surfactant system,
optionally in addition comprising at least one enzyme;
optionally further comprising 2-phenoxyethanol;
optionally further comprising 4,4′-dichloro 2-hydroxydiphenylether in a concentration from 0.001 to 3% by weight of the composition.

34. The composition of claim 33 being in liquid or semi-liquid form, further comprising at least one of i) at least one antimicrobial agent in an amount ranging from 2 ppm to 5% and ii) 4,4′-dichloro 2-hydroxydiphenylether in a concentration from 0.001 to 3%, each by weight of the composition.

35. A method of preserving a composition according to claim 32 against microbial contamination or growth, comprising adding 2-phenoxyethanol to the composition which is an aqueous composition comprising water as a solvent.

36. A method of laundering fabric or of cleaning hard surfaces, comprising treating a fabric or a hard surface with a composition according to claim 32, wherein the composition comprises 4,4′-dichloro 2-hydroxydiphenylether.
